(19) United States
(12) Patent Application Publication
(10) Pub. No.: US 2023/0295561 A1
(43) Pub. Date: Sep. 21, 2023

(54) LIQUID CRYSTAL SCAFFOLDS AND USE THEREOF

(71) Applicants: Paul WEISS, Los Angeles, CA (US); Ali TAMAYOL, Lincoln, NE (US); Amir NASAJPOUR, Los Angeles, CA (US); THE REGENTS OF THE UNIVERSITY OF CALIFORNIA, Oakland, CA (US); Board of Regents of the University of Nebraska, Lincoln, NE (US)

(72) Inventors: Paul Weiss, Los Angeles, CA (US); Ali Tamayol, Lincoln, NE (US); Amir Nasajpour, Los Angeles, CA (US)

(21) Appl. No.: 18/006,321

(22) PCT Filed: Jul. 23, 2021
(86) PCT No.: PCT/US2021/043029

§ 371 Date: Jan. 20, 2023

Related U.S. Application Data
(60) Provisional application No. 63/055,671, filed on Jul. 23, 2020.

(51) Int. Cl. C12N 5/00; C09K 19/36

(52) U.S. Cl. CPC C12N 5/0068; C09K 19/36; C12N 2533/20

(57) ABSTRACT

The present invention provides liquid crystals and compositions thereof (e.g., liquid crystal-based scaffolds). The present invention also provides the methods for generating said liquid crystals and compositions thereof (e.g., liquid crystal-based scaffolds) as well as uses thereof in cell-culturing and tissue-generating.

### CROSS-REFERENCE TO RELATED APPLICATIONS

This application claims priority to and the benefit of U.S. Provisional Application No. 63/055,671, filed Jul. 23, 2020, the disclosure of which is incorporated herein by reference in its entirety.

### BACKGROUND OF THE INVENTION

The liquid crystalline (LC) phase shares properties seen in both liquids and solids. Historically, LC-based materials have been applied in commercial applications with great success, such as in the development of body armor KEVLAR® and the fabrication of modern liquid crystal displays (Andrienko D et al., 2018, J. Mol. Liq., 267:520-541). More recently, LCs have been used to mimic various biological processes, ranging from epithelial tissue organization, bacterial biofilm formation, and the assembly of many biologically derived materials (Saw TB et al., 2017, Nature, 544:212-216; Pérez-Gonzalez C et al., 2019, Nat. Phys., 15:79-88; Patteson A E et al., 2018, Nat. Commun., 9:5373; Mitov M et al., 2017, Soft Matter, 13:4176-4209; Jewell S A et al., 2011, Liq. Cryst., 38:1699-1714). The synthesis of LC biomaterials is poised to address challenges in recapitulating mechanics seen in the native extracellular matrix (ECM) (Tibbitt M W et al., 2017, Acc. Chem. Res., 50:508-513; Martella D et al., 2018, Chem.: Eur. J., 24:12206-12220; Mohamed M A et al., 2019, Prog. Polym. Sci., 98:101147). An associated challenge for engineering LCs into tissue engineering substrates is the cytotoxicity associated with most commercially available LC mesogens.

Thus, there is a need in the art for methods and technologies that produce liquid-crystal-based biomaterials for the development of dynamic and responsive interfaces for tissue engineering. The present invention satisfies this unmet need.

### SUMMARY OF THE INVENTION

In one aspect, the present invention provides a method of inducing cell-culturing. In various embodiments, the method comprises culturing at least one cell on a liquid crystal or a composition thereof. In some embodiments, the method comprises culturing the at least one cell on a surface of the liquid crystal or the composition thereof.

In one embodiment, the cell is a tissue cell. In some embodiments, the cell is selected from the group consisting of an epithelial cell, endothelial cell, muscle cell, neuron, adipocyte, and any combination thereof.

In some embodiments, the liquid crystal comprises at least one selected from the group consisting of cholesteryl oleyl carbonate, cholesteryl pelargonate, and cholesteryl benzoate. In one embodiment, the liquid crystal is a cholesteryl ester liquid crystal.

In some embodiments, the composition comprises at least one selected from the group consisting of a polymer, solvent, composite, substrate, and additive.

In one embodiment, the polymer is a fibrous network. In some embodiments, the polymer is selected from the group consisting of an edible polymer, food grade polymer, biodegradable polymer, biocompatible polymer, and any combination thereof. In some embodiments, the polymer is selected from the group consisting of polyester, polycaprolactone, poly(ethylene glycol), and any combination thereof. In one embodiment, the polymer is polycaprolactone. In some embodiments, the composition comprises between about 5% to about 75% (w/v) polycaprolactone.

In one embodiment, the composition comprises the polymer and the solvent and wherein the composition is generated by dispersing the liquid crystal and the polymer in the solvent. In one embodiment, the composition is generated by dispersing the liquid crystal and the polymer in the solvent; and electrospinning the liquid crystal with the polymer.

In one embodiment, the composition is a cholesteryl ester liquid crystal-based scaffold. In one embodiment, the composition is a nonwoven cholesteryl ester liquid crystal-based scaffold. In some embodiments, the composition comprises between about 25% (w/v) to about 50% (w/v) cholesteryl ester liquid crystal.

In some embodiments, the composition is at least one selected from the group consisting of a composition having a mesophase between about 36° C. and about 40° C.; and a composition forming striations at a temperature between about 36° C. and about 40° C.

In some embodiments, the composition is selected from the group consisting of a pharmaceutical composition, edible composition, scaffold, and any combination thereof.

In some embodiments, the liquid crystal or the composition thereof is generated via a solvent-free method, polymer-free method, or a combination thereof.

In another aspect, the present invention relates, in part, to a method of exercising at least one cultured cell on a liquid crystal or a composition thereof through a treadmill action. In one embodiment, the method comprises a stimulus. In some embodiments, the stimulus is selected from the group consisting of a light stimulus, electrical stimulus, and mechanical stimulus.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 depicts representative cholesteryl ester liquid crystal scaffolds (CLC-S) composition used in Example 1. To generate the cholesteryl ester liquid crystal (CLC) mixtures for the electrospun scaffolds, reported CLC compositions were dispersed for the varying tested CLC-S conditions in a 4 mL azeotropic solvent mixture of chloroform:dimethylformamide (3:1).

FIG. 2 depicts a schematic representation of chemical structures of the compounds used for the synthesis of the cholesteryl ester liquid crystal scaffolds.

FIG. 3, comprising FIG. 3A through FIG. 3C, depicts representative results for microscopic investigation of CLC-S. FIG. 3A depicts a schematic representation of fabrication for the electrospun CLC-S: A 15% weight per volume (w/v) polycaprolactone (PCL) solution with cholesteryl ester liquid crystal mesogens of varying concentrations were electrospun. Scale bar is 5 mm. FIG. 3B depicts representative scanning electron microscopy images of the PCL-25 only scaffold, 25% (w/v) CLC-S, 37% (w/v) CLC-S, 50% (w/v) CLC-S. Scale bars are 100 μm. FIG. 3C depicts representative results of polarized optical micrograph (POM) imaging of the CLC-S condition and accompanying frequency-intensity plots. Scale bars are 200 μm.

FIG. 4, comprising FIG. 4A through FIG. 4C, depicts representative micrograph images of the engineered CLC-S. FIG. 4A depicts representative polycaprolactone (PCL) sample A′ and 50% (w/v) CLC-S sample B′ that were heated to 37° C. before the image was taken. It was evident from the micrographs that the CLC S sample showed a thermochromic property while the control PCL sample was opaque. FIG. 4B depicts representative results of an ultraviolet—visible spectroscopy of generated PCL, cholesteryl ester liquid crystal, and CLC-S at 37° C. FIG. 4C depicts that upon heating the CLC S to 37° C., the fingerprint-like striations can reflect light as seen in micrograph FIG. 4A.

FIG. 5 depicts representative scanning electron micrograph (SEM) of the CLC-S.

Varying magnification of the generated weight per volumes of 0%, 25%, 37%, and 50% CLC-S. PCL: polycaprolactone, CLC-S: cholesteryl ester liquid crystal scaffold.

FIG. 6, comprising FIG. 6A through FIG. 6D, depicts representative results of wide angle X-ray diffraction (WAXD) patterns and hydrophilicity of CLC-S. FIG. 6A depicts representative fiber diffraction patterns obtained for the scaffolds with varying cholesteryl ester liquid crystal concentrations. Broad reflection bands are annotated with black arrows within the micrographs of the CLC-S, showing a gradual move to higher θ and reflection sharpening. FIG. 6B depicts representative results of two-dimensional scattering intensity versus 2θ for each scaffold. FIG. 6C depicts representative contact angle micrographs for each scaffold. Scale bars are 1 mm. FIG. 6D depicts a plot of representative measured contact angle values for each scaffold. The contact angles of the PCL-only scaffold, 25% (w/v) CLC-S, and 37% (w/v) CLC-S were found to be significantly different from the 50% (w/v) CLC-S (n=6 for contact angle measurements, ***: P<0.001).

FIG. 7, comprising FIG. 7A through FIG. 7C, depicts representative results of surface and mechanical characterization of the CLC-S. FIG. 7A depicts representative force-distance curves of the tested scaffolds with atomic force microscopy (AFM). FIG. 7B depicts representative nanoindentation using AFM showing that the Young's moduli of the scaffolds decreased with increasing concentration of CLC. The 50% (w/v) CLC-S displayed Young's moduli on the kPa scale, three orders of magnitude lower than that of the PCL only scaffold. FIG. 7C depicts representative results of temperature controlled atomic force microscopy phase contrast imaging of depicts fingerprint-like striation conditions at the engineered phase transition temperature. The 50% (w/v) CLC-S was heated to 37° C. then cooled to room temperature.

FIG. 8, comprising FIG. 8A and FIG. 8B, depicts representative results from temperature ramp experiments. FIG. 8A depicts representative atomic force microscopy (AFM) assessment of temperature ramp experiments with 50% (w/v) CLC-S. Upper panels are topographic images; lower panels are simultaneously acquired phase contrast images of the same area. FIG. 8B depicts representative samples that were heated to 37° C. and monitored over an hour. This measurement indicated that the CLC domains were dynamic and moving within the webbing of the nanofibers. CLC-S: cholesteryl ester liquid crystal scaffold.

FIG. 9, comprising FIG. 9A and FIG. 9B, depicts representative results from temperature ramp experiments. FIG. 9A depicts representative AFM assessment of temperature ramp experiments with 50% (w/v) CLC-S. Upper panels are topographic images; lower panels are simultaneously acquired phase contrast images of the same area. FIG. 9B depicts representative samples that were heated to 37° C. then cooled to room temperature. The appearance of CLC domains within the webbing of the scaffolds was found to be reversible. CLC-S: cholesteryl ester liquid crystal scaffold.

FIG. 10, comprising FIG. 10A through FIG. 10D, depicts representative results of bulk mechanical testing of CLC-S. FIG. 10A depicts representative stress-strain curve for generated hybrid CLC-S. FIG. 10B depicts representative tensile strength for generated hybrid CLC-S. FIG. 10C depicts representative ultimate strain for generated hybrid CLC-S. FIG. 10D depicts representative Young's modulus for generated hybrid CLC-S (n=4, statistical analysis by one-way ANOVA).

FIG. 11, comprising FIG. 11A through FIG. 11D, depicts representative results demonstrating in vitro biocompatibility of muscle cells with CLC-S. FIG. 11A depicts representative results of presto blue analysis (metabolic analysis) of the mouse myoblast cells (C2C12) cultured over 7 days. FIG. 11B depicts representative atomic force microscopy measurements of C2C12 cellular stiffness on tested scaffolds. FIG. 11C representative results of immunohistochemistry staining of myosin heavy chain (MEW) and 4′,6-diamidino-2-phenylindole (DAPI) of C2C12s cultured on 50% (w/v) CLC-S scaffold compared to positive control Day 14. White arrow highlights multinucleated phenotype of mouse myoblast. Scale bars are 100 μm. FIG. 11D depicts representative scanning electron microscopy images of cultured C2C12s on scaffolds Day 14. Scale bars are 100 μm in the upper panels and 30 μm in the lower panels. (n=6 for C2C12 metabolic activity assays, n=4 for elastic modulus measurements, **: P<0.01, ***: P<0.001.

FIG. 12 depicts representative live/dead staining of the CLC-S. Samples were incubated with calcein acetoxymethyl ester and ethidium homodimer in phosphate buffered saline and imaged under fluorescence microscope.

FIG. 13 depicts representative phase contrast images of mouse myoblast (C2C12) cells cultured on the substrates generated from atomic force microscopy experiments.

FIG. 14 depicts representative F-actin/DAPI staining of cells cultured on the CLC-S on day 7 (before differentiation) and day 14 (after differentiation). DAPI: 4′,6-diamidino-2-phenylindole.

FIG. 15 depicts representative scanning electron micrograph (SEM) of the CLC-S day 14 differentiation of mouse myoblast (C2C12). PCL: polycaprolactone

FIG. 16 depicts a schematic representation of CLC cell-laden microcarriers for bioreactor culture.

FIG. 17 depicts representative Corning Bioreactor (10-micron polymer beads (polystyrene beads).

FIG. 18 depicts representative CLC-modified Corning Bioreactor polymer beads.

FIG. 19 depicts representative non-modified Corning Bioreactor (10-micron polymer beads (polystyrene beads) (left) compared to representative CLC-modified Corning Bioreactor polymer beads (right).

FIG. 20 depicts representative unstained day 1 mouse myoblast cells (C2C12) that self-assembled spontaneously into 3D muscle spheroids on thick CLC coating conditions highlighted in white.

FIG. 21 depicts representative stained day 1 mouse myoblast cells (C2C12) self-assembled spontaneously into 3D muscle spheroids on thick CLC coating conditions highlighted in white.

FIG. 22 depicts representative stained day 1 mouse myoblast cells (C2C12) cultured on a thin micron layer of CLC. The cells consume the CLC under culture and spread back into normal 2D spindle elongated structures however some traces of CLC retain the phenotypical 3D muscle spheroids on the not consumed thick CLC coating conditions highlighted in white.

FIG. 23 depicts representative stained day 1 mouse myoblast cells (C2C12) cultured on a thick millimeter layer of CLC. The cell at the interface of the CLC spread in typical 2D spindle elongated structures however myoblast cells seeded on thick layer of the CLC have a 3D muscle spheroids phenotype highlighted in white.

FIG. 24 depicts representative digital photographs taken during a 24 h observation of human induced pluripotent stem cells (iPSC) with CLC.

FIG. 25 depicts representative heart of palm (left) compared to representative decellularized heart of palm (right).

FIG. 26 depicts representative decellularized heart of palm coated with CLC (w/w % Light) (left) compared to representative decellularized heart of palm (right).

FIG. 27 depicts representative decellularized heart of palm coated with CLC (w/w % Heavy).

### DETAILED DESCRIPTION

The present invention is based, in part, on the discovery of novel methods of inducing growth of muscle cells using cholesteryl ester liquid crystals or cholesteryl ester liquid crystal scaffolds. Thus, the present invention is directed, in part, to methods of inducing cell-culturing using a liquid crystal or a composition thereof (e.g., liquid crystal scaffold). The present invention is also directed, in part, to methods of generating at least one cell tissue layer using a liquid crystal or a composition thereof (e.g., liquid crystal scaffold). In one aspect, the methods of the present invention relate to inducing cell-culturing or generating at least one cell tissue layer of a tissue cell. In other aspects, the methods of the present invention relate to inducing cell-culturing or generating at least one cell tissue layer of an epithelial cell, endothelial cell, muscle cell, neuron, adipocyte, or any combination thereof. For example, in one embodiment, the cell is a muscle cell. Thus, in some embodiments, the liquid crystal or a composition thereof is used in food applications (e.g., meat growth) or medical applications (e.g., wound healing, coatings, etc.).

In some aspects of the invention, the liquid crystal comprises cholesteryl oleyl carbonate, cholesteryl pelargonate, cholesteryl benzoate, or any combination thereof. For example, in one embodiment, the liquid crystal is a cholesteryl ester liquid crystal. In other aspects of the invention, the composition further comprises a polymer, solvent, additive, or any combination thereof. In some embodiments, the composition is generated by dispersing the liquid crystal and the polymer in the solvent and electrospinning the liquid crystal with the polymer.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass non-limiting variations of ±40% or ±20% or ±10%, ±5%, ±1%, or ±0.1% from the specified value, as such variations are appropriate.

The term “derivative” refers to a small molecule that differs in structure from the reference molecule, but retains the essential properties of the reference molecule. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

As used herein, the terms “material” and “materials” refer to, in their broadest sense, any composition of matter.

As used herein, the term “substrate” refers to a solid object or support upon which another material is layered or attached. Solid supports include, but are not limited to, glass, metals, gels, and filter paper, among others.

As used herein, the term “mesogen” refers compounds that form liquid crystals, and in particular rigid rodlike or disclike molecules which are components of liquid crystalline materials.

As used herein, the term “liquid crystal” refers to a thermodynamic stable phase characterized by anisotropy of properties without the existence of a three-dimensional crystal lattice, generally lying in the temperature range between the solid and isotropic liquid phase.

As used herein, “thermotropic liquid crystal” refers to liquid crystals that result from the melting of mesogenic solids due to an increase in temperature. Both pure substances and mixtures form thermotropic liquid crystals.

The term “lyotropic,” as used herein, refers to molecules that form phases with orientational and/or positional order in a solvent. Lyotropic liquid crystals can be formed using amphiphilic molecules (e.g., sodium laurate, phosphatidylethanolamine, lecithin). The solvent can be water.

As used herein, the term “heterogenous surface” refers to a surface that orients liquid crystals in at least two separate planes or directions, such as across a gradient.

As used herein, “nematic” refers to liquid crystals in which the long axes of the molecules remain substantially parallel, but the positions of the centers of mass are randomly distributed. Nematic liquid crystals can be substantially oriented by a nearby surface.

“Chiral nematic,” as used herein refers to liquid crystals in which the mesogens are optically active. Instead of the director being held locally constant, as is the case for nematics, the director rotates in a helical fashion throughout the sample. Chiral nematic crystals show a strong optical activity, which is much higher than can be explained on the basis of the rotatory power of the individual mesogens. When light equal in wavelength to the pitch of the director impinges on the liquid crystal, the director acts like a diffraction grating, reflecting most and sometimes all of the light incident on it. If white light is incident on such a material, only one color of light is reflected and it is circularly polarized. This phenomenon is known as selective reflection and is responsible for the iridescent colors produced by chiral nematic crystals.

“Smectic,” as used herein refers to liquid crystals that are distinguished from “nematics” by the presence of a greater degree of positional order in addition to orientational order; the molecules spend more time in planes and layers than they do between these planes and layers. “Polar smectic” layers occur when the mesogens have permanent dipole moments. In the smectic A2 phase, for example, successive layers show anti ferroelectric order, with the direction of the permanent dipole alternating from layer to layer. If the molecule contains a permanent dipole moment transverse to the long molecular axis, then the chiral smectic phase is ferroelectric. A device utilizing this phase can be intrinsically bistable.

“Frustrated phases,” as used herein, refers to another class of phases formed by chiral molecules. These phases are not chiral, however, twist is introduced into the phase by an array of grain boundaries. A cubic lattice of defects (where the director is not defined) exist in a complicated, orientationally ordered twisted structure. The distance between these defects is hundreds of nanometers, so these phases reflect light just as crystals reflect X-rays.

“Discotic phases” are formed from molecules which are disc shaped rather than elongated. Usually these molecules have aromatic cores and six lateral substituents. If the molecules are chiral, a chiral nematic discotic phase can form.

As used herein, the term “transparent” may refer to a material that permits at least 50% of the incident electromagnetic radiation at relevant wavelengths to be transmitted through it. In a device comprising a liquid crystal surface of the present invention, it may be desirable to allow the maximum amount of ambient electromagnetic radiation from the device exterior to be admitted to the liquid crystal layer region of the device. That is, the electromagnetic radiation must reach a liquid crystal layer(s), where it can stimulate the color change of the liquid crystal layer. This often dictates that at least one of the substrates of the device should be minimally absorbing and minimally reflecting of the incident electromagnetic radiation. In some cases, such a contact should be transparent or at least semi-transparent.

As used herein, the term “semi-transparent” may refer to a material that permits some, but less than 50% transmission of ambient electromagnetic radiation in relevant wavelengths. Where a transparent or semi-transparent substrate is used, the opposing substrate may be a reflective material so that light which has passed through the liquid crystal layer without being stimulating the color change of the liquid crystal layer is reflected back through the liquid crystal layer.

As used herein, the term “polymer” refers to a molecule composed of repeating structural units typically connected by covalent chemical bonds. The term “polymer” is also meant to include the terms copolymer and oligomers. In one embodiment, a polymer comprises a backbone (i.e., the chemical connectivity that defines the central chain of the polymer, including chemical linkages among the various polymerized monomeric units) and a side chain (i.e., the chemical connectivity that extends away from the backbone).

As used herein, “surface”, “top surface”, and “external surface” of the substrate are used interchangeably and refer to the surface of the substrate furthest away from the liquid crystal layer, while “bottom surface” or “internal surface” of the substrate are used interchangeably and refer to the surface of the substrate closest to the liquid crystal layer. Where a first layer is described as “disposed over” a second layer, the first layer is disposed further away from the external surface of the substrate. There may be other layers between the first and second layer, unless it is specified that the first layer is “in contact with” the second layer. For example, a liquid crystal layer may be described as “disposed over” at least a portion of the internal surface of the substrate, even though there are various organic layers in between.

As used and depicted herein, a “layer”, for example a liquid crystal layer, refers to a member or component of a device being principally defined by a thickness, for example in relation to other neighboring layers, and extending outward in length and width. It should be understood that the term “layer” is not necessarily limited to single layers or sheets of materials. In addition, it should be understood that the surfaces of certain layers, including the interface(s) of such layers with other material(s) or layers(s), may be imperfect, wherein said surfaces represent an interpenetrating, entangled or convoluted network with other material(s) or layer(s). Similarly, it should also be understood that a layer may be uniform or discontinuous, such that the continuity of said layer along the length and width may be disturbed or otherwise interrupted by other layer(s) or material(s).

The terms “coat,” “coated,” or “coating,” as used herein, refer to at least a partial coating of the surface of the substrate. One hundred percent coverage is not necessarily implied by these terms.

As used herein, “spin coating” may refer to the process of solution depositing a layer or film of one material (i.e., the coating material) on a surface of the device. The spin coating process may include applying a small amount of the coating material on the center of the substrate, which is either spinning at low speed or not spinning at all. The substrate is then rotated at high speed in order to spread the coating material by centrifugal force. Rotation is continued while the fluid spins off the edges of the substrate, until the desired thickness of the film is achieved. The higher the angular speed of spinning, the thinner the film. The thickness of the film also depends on the viscosity of the liquid crystal mesogen.

The terms “electrospinning” or “electrospun”, as used herein refer to any method where materials are streamed, sprayed, sputtered, dripped, or otherwise transported in the presence of an electric field. The electrospun material can be deposited from the direction of a charged container towards a grounded target, or from a grounded container in the direction of a charged target. In particular, the term “electrospinning” means a process in which fibers are formed from a charged solution comprising at least one natural biological material, at least one synthetic polymer material, or a combination thereof by streaming the electrically charged solution through an opening or orifice towards a grounded template. As used herein, the terms “solution”, “solvent”, and “fluid” refer to a liquid that is capable of being charged and which comprises at least one natural material, at least one synthetic polymer, or a combination thereof.

The term “scaffold”, as used herein, refers to any material that allows attachment of cells, preferably attachment of cells involved in meat growth or wound healing.

“Attachment”, “attach” or “attaches” as used herein, refers to cells that adhere directly or indirectly to a substrate as well as to cells that adhere to other cells. Preferably the scaffold is three dimensional.

As used herein, the term “stem cell niche” refers to a cavity within an electrospun scaffold capable of housing one or more cells, e.g., stem cells, therein and providing a sheltering environment that physically protects said cells from physical disturbance and/or from stimulus that may promote differentiation and apoptosis. Preferably, the niche is a cavity defined by a concave surface within an electrospun scaffold, for example in the form of a pocket, a recess, a groove or a ridge.

As used herein, the term “spectrum” refers to the distribution of light energies arranged in order of wavelength.

As used the term “visible spectrum” refers to light radiation that contains wavelengths from approximately 360 nm to approximately 800 nm.

As used herein, the term “ultraviolet irradiation” refers to exposure to radiation with wavelengths less than that of visible light (i.e., less than approximately 360 nm) but greater than that of X-rays (i.e., greater than approximately 0.1 nm). Ultraviolet radiation possesses greater energy than visible light and is therefore, more effective at inducing photochemical reactions.

As used herein, the term “solvent” describes a liquid that serves as the medium for a reaction or a medium for the distribution of components of different phases or extraction of components into said solvent. Also, as used herein, the term “solvent” is intended to encompass liquids in which the raw materials or the reaction mixture are dispersed, suspended, or at least partially solvated. Examples of solvents include, but are not limited to, alcohols, ethers, acetones, DMSO, DMF, benzene, toluene, chloroform, dichloromethane, and hexanes.

As used herein, the terms “solvent-free”, “at least substantially solvent-free”, “at least substantially free of a solvent”, and other like variants are used interchangeably to mean that no solvent is intentionally added to, or used in, any raw material or the reaction mixture (which includes all of the raw materials) during any of the processing steps leading to the formation of the metallic silver. It is to be understood that a raw material or reaction mixture that is at least substantially free of a solvent may inadvertently contain small amounts of a solvent owing to contamination or it may contain no amount of solvent.

As used herein the term “degradation” relates to the breakdown of the polymer structure of the scaffold. This breakdown of structural integrity is accompanied by the release from the scaffold of degradation products from the polymer and a reduction in the mechanical strength of the scaffold.

As used herein, the term “biodegradable” refers to material or polymer that can be degraded, preferably adsorbed and degraded in a patient's body. Preferably the scaffold is biodegradable, i.e., is formed of biodegradable materials, such as biodegradable polymers or naturally occurring biological materials.

As used herein, the terms “biocompatible” and “biologically compatible” are used interchangeably to the ability of a material, i.e., a polymer, to be implanted into or be administered to a human or animal body, without eliciting any undesirable local or systemic effects in the recipient, for example, without eliciting significant inflammation and/or acute rejection of the polymer by the immune system, for instance, via a T-cell response.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

The terms “patient,” “subject,” “individual,” and the like are used interchangeably herein, and refer to any animal, or cells thereof whether in vitro or in situ, amenable to the methods described herein. In some non-limiting embodiments, the patient, subject or individual is a human. In various embodiments, the subject is a human subject, and may be of any race, sex, and age.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based, in part, on the discovery of novel methods of inducing growth of muscle cells using cholesteryl ester liquid crystals or cholesteryl ester liquid crystal scaffolds. Thus, the present invention is directed, in part, to methods of inducing cell-culturing using a liquid crystal or a composition thereof (e.g., liquid crystal scaffold). The present invention is also directed, in part, to methods of generating at least one cell tissue layer using a liquid crystal or a composition thereof (e.g., liquid crystal scaffold). In one aspect, the methods of the present invention relate to inducing cell-culturing or generating at least one cell tissue layer of a tissue cell. In other aspects, the methods of the present invention relate to inducing cell-culturing or generating at least one cell tissue layer of an epithelial cell, endothelial cell, muscle cell, neuron, adipocyte, or any combination thereof. For example, in one embodiment, the cell is a muscle cell. Thus, in some embodiments, the liquid crystal or a composition thereof is used in food applications (e.g., meat, such as beef, pork, fish, etc., growth) or medical applications (e.g., wound healing, muscle repair, coatings, etc.).

### Liquid Crystals and Compositions Thereof

In one aspect, the present invention relates, in part, to a liquid crystal or a composition thereof for inducing cell-culturing and/or generating at least one cell tissue layer. Examples of such cell include, but are not limited to a tissue cell, epithelial cell, endothelial cell, muscle cell, neuron, adipocyte, stem cell (e.g., a muscle stem cell, mesenchymal stem cell, epithelial stem cell, etc.), fat cells, or any combination thereof

For example, in various aspects, the liquid crystal or the composition thereof of the present invention is a liquid crystal or a composition thereof for inducing cellular agriculture of at least one cell or cell tissue layer of interest (e.g., bovine cell, avian cell, such chicken cell, duck cell, turkey cell, quail cell, etc., swine/porcine cell, sheep cell, goat cell, piscine cell, such as tuna cell, salmon cell, snapper cell, cod cell, etc., shellfish cell, such as lobster cell, crab cell, shrimp cell, crayfish cell, clam cell, oyster cell, mussel cell, etc., or any combination thereof).

In other embodiments, the liquid crystal or the composition thereof of the present invention is a liquid crystal or a composition thereof for inducing and/or propagating cellular agriculture of at least one cell or cell tissue layer of interest (e.g., human organ, cancer spheroids for drug testing, etc.).

In various embodiments, the liquid crystal comprises cholesteryl oleyl carbonate or a derivative or a salt thereof, cholesteryl pelargonate or a derivative or a salt thereof, cholesteryl benzoate or a derivative or a salt thereof, or any combination thereof. Thus, in one embodiment, the liquid crystal comprises a cholesteryl ester liquid crystal. In another embodiment, the liquid crystal is a cholesteryl ester liquid crystal.

In some embodiments, the liquid crystal comprises between about 50 mg to about 2000 mg cholesteryl oleyl carbonate or a derivative or a salt thereof, between about 50 mg to about 2000 mg cholesteryl pelargonate or a derivative or a salt thereof, between about 50 mg to about 2000 mg cholesteryl benzoate or a derivative or a salt thereof, or any combination thereof. For example, in one embodiment, the liquid crystal comprises about 320 mg cholesteryl oleyl carbonate or a derivative or a salt thereof, about 580 mg cholesteryl pelargonate or a derivative or a salt thereof, about 100 mg cholesteryl benzoate or a derivative or a salt thereof, or any combination thereof. In another embodiment, the liquid crystal comprises about 480 mg cholesteryl oleyl carbonate or a derivative or a salt thereof, about 870 mg cholesteryl pelargonate or a derivative or a salt thereof, about 150 mg cholesteryl benzoate or a derivative or a salt thereof, or any combination thereof. In yet another embodiment, the liquid crystal comprises about 640 mg cholesteryl oleyl carbonate or a derivative or a salt thereof, about 1160 mg cholesteryl pelargonate or a derivative or a salt thereof, about 200 mg cholesteryl benzoate or a derivative or a salt thereof, or any combination thereof.

In other embodiments, the liquid crystal comprises synthetic polyurethane lacquer, natural lacquer derived from catechol molecules, urushiol mixtures, or any combination thereof.

In some embodiments, the liquid crystal has a mesophase between about 10° C. and about 60° C. In some embodiments, the liquid crystal has a mesophase between about 17° C. and about 40° C. In some embodiments, the liquid crystal has a mesophase between about 36° C. and about 40° C. In some embodiments, the liquid crystal has a mesophase at about 37° C.

In some embodiments, the liquid crystal forms striations between about 10° C. and about 60° C. In some embodiments, the liquid crystal forms striations at a temperature between about 17° C. and about 40° C. In some embodiments, the liquid crystal forms striations at a temperature between about 36° C. and about 40° C. In some embodiments, the liquid crystal forms striations at a temperature about 37° C.

In various embodiments, the liquid crystal comprises a mesogenic layer. In one embodiment, the liquid crystal comprises a mesogenic layer comprising cholesteryl oleyl carbonate or a derivative or a salt thereof, cholesteryl pelargonate or a derivative or a salt thereof, cholesteryl benzoate or a derivative or a salt thereof, or any combination thereof. In another embodiment, any compound or mixture of compounds that form a mesogenic layer can be used in conjunction with the present invention.

The mesogens can form thermotropic or lyotropic liquid crystals. The mesogenic layer can be either continuous or it can be patterned. Both the thermotropic and lyotropic liquid crystals can exist in a number of forms including nematic, chiral nematic, smectic, polar smectic, chiral smectic, frustrated phases and discotic phases.

The mesogenic layer can be a substantially pure compound, or it can contain other compounds that enhance or alter characteristics of the mesogen. Thus, in one embodiment, the mesogenic layer further comprises a second compound, for example an alkane, which expands the temperature range over which the nematic and isotropic phases exist.

In some embodiments, the mesogenic layer further comprises a dichroic dye or fluorescent compound. Examples of dichroic dyes and fluorescent compounds useful in the present invention include, but are not limited to, azobenzene, BTBP, polyazocompounds, anthraquinone, perylene dyes, and the like. In some embodiments, a dichroic dye of fluorescent compound is selected that complements the orientation dependence of the liquid crystal so that polarized light is not required for the device of the present invention to show different colors. In some embodiments, if the absorbance of the liquid crystal is in the visible range, then changes in orientation can be observed using ambient light without crossed polarizers. In other embodiments, the dichroic dye or fluorescent compound is used in combination with a fluorimeter and the changes in fluorescence are used to detect changes in orientation of the liquid crystal.

In various embodiments, the liquid crystal is a molecular switch. In one embodiment, the liquid crystal changes color when exposed to a stimulus. In various embodiments, the mesogenic layers of the instant invention can be tuned by the use of at least one stimulus. In some embodiments, the stimulus comprises applying energy or a pH change to the device. Examples of such stimulus include, but are not limited to temperature, electric field (e.g., voltage), electromagnetic field, magnetic field, light, optical methods (e.g., ultraviolet (UV) irradiation, UV-vis-NIR irradiation, infrared (IR) irradiation, NIR irradiation), radiofrequencies, radiation, sound, hydration, pH, pressure, or any combination thereof.

In one embodiment, the stimulus is used to reversibly orient the mesogenic layer. In one embodiment, the stimulus is applied either perpendicular to, or in the plane of, the surface of the mesogenic layer. In one embodiment, the oriented mesogenic layer modulates the intensity of light diffracted from the layer.

For example, in one embodiment the mesogenic layers of the instant invention can be tuned by the use of electric fields. In one embodiment, the electric field is used to reversibly orient the mesogenic layer. In one embodiment, the electric field is applied either perpendicular to, or in the plane of, the surface of the mesogenic layer. In one embodiment, the oriented mesogenic layer modulates the intensity of light diffracted from the layer.

In another embodiment the mesogenic layers of the instant invention can be tuned by the use of temperature (e.g., heat). In one embodiment, the temperature (e.g., heat) is used to reversibly orient the mesogenic layer. In one embodiment, the temperature (e.g., heat) is applied either perpendicular to, or in the plane of, the surface of the mesogenic layer. In one embodiment, the oriented mesogenic layer modulates the intensity of light diffracted from the layer.

In another embodiment, the mesogenic layer is subsequently cooled to form the liquid crystalline phase. The presence of the stimulus within regions of the mesogenic layer will disturb the equilibrium between the nematic and isotropic phases leading to different rates and magnitudes of nucleation at those sites. The differences between the nematic and isotropic regions are clearly detectable.

When the liquid crystal is exposed to the stimulus, the orientation of the liquid crystal is disrupted. In some embodiments, the disruption of orientation can be detected by a variety of methods, including detecting a color change of the liquid crystal, viewing with crossed polarizers, measuring the threshold electrical field required to change the orientation of the liquid crystal, viewing in the presence of dichroic agents, or any combination thereof. The liquid crystals can be viewed using white light or using a specific wavelength or combination of wavelengths of light.

Although many changes in the mesogenic layer can be detected by visual observation under ambient light, any means for detecting the change in the mesogenic layer can be incorporated into, or used in conjunction with, the device. Thus, it is within the scope of the present invention to use lights, microscopes, spectrometry, electrical techniques and the like to aid in the detection of a change in the mesogenic layer.

In those embodiments utilizing light in the visible region of the spectrum, the light can be used to simply illuminate details of the mesogenic layer. Alternatively, the light can be passed through the mesogenic layer and the amount of light transmitted, absorbed or reflected can be measured. The device can utilize a backlighting device such as that described in U.S. Pat. No. 5,739,879, incorporated herein by reference. Light in the ultraviolet and infrared regions is also of use in the present invention.

The present invention also relates, in part, to the use of plate readers to detect changes in the orientation of mesogens. The plate readers may be used in conjunction with the LC assay devices described herein and also with the lyotropic LC assays described in U.S. Pat. No. 6,171,802, incorporated herein by reference. In particular, the present invention includes methods and processes for the quantification of light transmission through films of liquid crystals based on quantification of transmitted or reflected light.

In other aspects, the present invention relates, in part, to liquid crystal compositions for inducing cell-culturing and/or generating at least one cell tissue layer. In various embodiments, the composition comprises any liquid crystal described herein. For example, in some embodiments, the composition comprises a liquid crystal layer. In some embodiments, the composition comprises a uniformly oriented liquid crystal.

In various embodiments, the composition is a tunable composition. In some embodiments, the tunable composition permits the manipulation of light. In one embodiment, the composition is a refractive-diffractive device. In one embodiment, the composition permits imaging from a single optical element. In one embodiment, the composition permits aplanatic or chromatic correction in lenses. In one embodiment, the composition allows for spectral dispersion. Thus, for example, in one embodiment, the tunable composition changes color when exposed to a stimulus.

In some embodiments, the composition comprises at least one cell. Examples of such cell include, but are not limited to a tissue cell, epithelial cell, endothelial cell, muscle cell, neuron, adipocyte, stem cell (e.g., a muscle stem cell, mesenchymal stem cell, epithelial stem cell, etc.), fat cells, or any combination thereof.

In some embodiments, the composition comprises a polymer, solvent, additive, substrate, composite, or any combination thereof.

In some embodiments, the composition comprises between about 5% (w/v) to between about 95% (w/v) liquid crystal. In some embodiments, the composition comprises between about 15 15% (w/v) to between about 85% (w/v) liquid crystal. In some embodiments, the composition comprises between about 25% (w/v) to between about 50% (w/v) liquid crystal. In one embodiment, the composition comprises about 25% (w/v) liquid crystal. In one embodiment, the composition comprises about 37% (w/v) liquid crystal. In one embodiment, the composition comprises about 50% (w/v) liquid crystal.

In some embodiments, the composition comprises between about 0.000001% (w/v) to between about 95% (w/v) polymer. In some embodiments, the composition comprises between about 15% (w/v) to between about 85% (w/v) polymer. In some embodiments, the composition comprises between about 15% (w/v) to between about 50% (w/v) polymer. In some embodiments, the composition comprises between about 0.000001% (w/v) to between about 1% (w/v) polymer. In one embodiment, the composition comprises about 15% (w/v) polymer.

In some embodiments, the composition comprises between about 5% (w/v) to between about 95% (w/v) solvent. In some embodiments, the composition comprises between about 15% (w/v) to between about 85% (w/v) solvent. In some embodiments, the composition comprises between about 25% (w/v) to between about 50% (w/v) solvent. In one embodiment, the composition comprises about 85% (w/v) solvent.

In some embodiments, the composition comprises between about 0.05% (w/v) to between about 95% (w/v) additive. In some embodiments, the composition comprises between about 1.5% (w/v) to between about 85% (w/v) additive. In some embodiments, the composition comprises between about 2.5% (w/v) to between about 50% (w/v) additive. In one embodiment, the composition comprises about 0.25% (w/v) additive. In one embodiment, the composition comprises about 3% (w/v) additive. In one embodiment, the composition comprises about 5% (w/v) additive.

In some embodiments, the composition comprises between about 5% (w/v) to between about 95% (w/v) substrate. In some embodiments, the composition comprises between about 15% (w/v) to between about 85% (w/v) substrate. In some embodiments, the composition comprises between about 15% (w/v) to between about 50% (w/v) substrate. In one embodiment, the composition comprises about 15% (w/v) substrate.

In some embodiments, the composition comprises between about 5% (w/v) to between about 95% (w/v) composite (e.g., resin matrix). In some embodiments, the composition comprises between about 15% (w/v) to between about 85% (w/v) composite. In some embodiments, the composition comprises between about 15% (w/v) to between about 50% (w/v) composite. In one embodiment, the composition comprises about 15% (w/v) composite.

In some embodiments, the solvent is a liquid in which the raw materials or the reaction mixture are dispersed, suspended, or at least partially solvated. Examples of solvents include, but are not limited to, alcohols, ethers, acetones, benzene, toluene, chloroform, dichloromethane, DMSO, and cyclohexanes.

In some embodiments, the composite is an organic-inorganic composite, nacre, glass composite, fiber composite, glass fiber composite, carbon composite, resin matrix, or any combination thereof.

In some embodiments, the polymer is a biodegradable polymer, biocompatible polymer, edible polymer, food grade polymer, or any combination thereof.

In some embodiments, the polymer has molecular weight of 5 kDa-3000 kDa. For example, in one embodiment, the polymer has a molecular weight of 5 kDa-2000 kDa, 5 kDa-1500 kDa, 5 kDa-1000 kDa, 5 kDa-800 kDa, 5 kDa-500 kDa, 5 kDa-300 kDa or 5 kDa-200 kDa or 800 kDa-3000 kDa.

In some embodiments, the polymer is a straight chain polymer (i.e., linear polymer) or a branched chain polymer (i.e., branched polymer), including hyperbranched polymers. In some embodiments, the polymer is cross-linked. In one embodiment, the polymer is a fibrous network.

In some embodiments, the polymer is a neutral polymer, ionic polymer, anionic polymer, or cationic polymer.

In some embodiments, the polymer is a homopolymer, copolymer, or block copolymer. In some embodiments, the block copolymer is a triblock, tetrablock, pentablock, or at least six block copolymer.

In some embodiments, the polymer is polyester, polyolefin, poly(vinyl chloride), polystyrene, polycaprolactone, polyethylene, polycarbonate, polyalkyleneimine (e.g., polyethyleneimine), polyallylamine, polyamidoamine, or poly(amino-co-ester), or any combination thereof. Examples of polymers also include, but are not limited to poly(ethylene oxide) (PEO) block copolymer, polyacrylate, polymethacrylate, polyamine, polyalkyleneimine (e.g., polyethyleneimine), polyallylamine, polyamidoamine, polyolefin, poly(amino-co-ester), poly(ethylethylene) (PEE), polymethyl methacrylate (PMMA), polyethyleneimine (PEI), chitosan, poly(butadiene) (PB or PBD), poly(styrene) (PS), poly(isoprene) (PI), polyethyleneimine (PEI), poly(lactide-co-glycolic acid) (PLGA), biodegradable PLGA, polyethylene glycol (PEG), poly(lactide-co-glycolic acid)-polyethylene glycol (PLGA-PEG), poly(lactide-co-glycolic acid)-block-polyethylene glycol (PLGA-b-PEG), biodegradable PLGA-PEG, biodegradable PLGA-b-PEG, polyanhydride, polyanhydride-block- PEG copolymers, zwitterionic poly(carbobetaine), zwitterionic poly(sulfobetaine)-containing, zwitterionic poly(carbobetaine) and zwitterionic poly(sulfobetaine)-containing copolymers, poly(acrylic acid-co-distearin acrylate), poly(trimethylene carbonate)-block-poly(L-gluatamic acid), poly(ethylene glycol-block-L-aspartic acid), poly(2-hydroxyethyl-co-octadecyl aspartamide), poly(ethylene glycol-co-trimethylene carbonate-co-caprolactone, polypropylene oxide block copolymers, polyethylene oxide-block-polypropylene oxide copolymers, poly(c-caprolactone) (PCL) diblock co-polymer, poly(ethylene oxide)-block-poly(c-caprolactone) (PEO-b-PCL) based diblock copolymers, poly(lactic acid), poly(glycolide), poly(lactic-coglycolic acid), poly(3-hydroxybutyrate), poly(lactide-co-glycolic acid)-polyethylene glycol (PLGA-PEG), poly(lactide-co-glycolic acid)-block-polyethylene glycol (PLGA-b-PEG), chitosan, poly(2-N,N-dimethylaminoethylmethacrylate), poly-L-lysine, zein, alginate, hyaluronic acid, mycelium, or any combination thereof. For example, in one embodiment, the polymer is polycaprolactone.

In one embodiment, the polymer is an organic polymer. Examples of such organic polymers include, but are not limited to, polyalkenes (e.g., polyethylene, polyisobutene, polybutadiene), polyacrylics (e.g., polyacrylate, polymethyl methacrylate, polycyanoacrylate), polyvinyls (e.g., polyvinyl alcohol, polyvinyl acetate, polyvinyl butyral, polyvinyl chloride), polystyrenes, polycarbonates, polyesters, polyurethanes, polyamides, polyimides, polysulfone, polysiloxanes, polyheterocycles, cellulose derivative (e.g., methyl cellulose, cellulose acetate, nitrocellulose), polysilanes, fluorinated polymers, epoxies, polyethers, phenolic resins (e.g., Cognard, J. Alignment of Nematic Liquid Crystals and Their Mixtures, in Mol. Cryst. Liq. Cryst. 1: 1-74 (1982)), polydimethylsiloxane, polyethylene, polyacrylonitrile, cellulosic materials, polycarbonates, polyvinyl pyridinium, zein, alginate, hyaluronic acid, mycelium, or any combination thereof.

In one embodiment, the polymer is a synthetic polymer. A synthetic polymer material can be any material prepared through a method of artificial synthesis, processing, or manufacture. Both the biological and polymeric materials are capable of being charged under an electric field.

In one embodiment, the polymer is a biocompatible polymer.

In some embodiments, the polymer is a biocompatible synthetic polymer. Examples of biocompatible synthetic polymers include, but are not limited to, poly(urethanes), poly(siloxanes) or silicones, poly(ethylene), poly(vinyl pyrrolidone), poly(-hydroxy ethyl methacrylate), poly(N-vinyl pyrrolidone), poly(methyl methacrylate), polyvinyl alcohol) (PVA), poly(acrylic acid), polyvinyl acetate), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, polyvinyl acetate), polyvinylhydroxide, zein, alginate, hyaluronic acid, mycelium, poly(ethylene oxide) (PEO) and polyorthoesters or co-polymers thereof. In some embodiments, the biocompatible polymer is PGLA.

In one embodiment, the polymer is a biodegradable polymer. Examples of suitable biodegradable materials include, but are not limited to collagen, poly(alpha esters) such as poly(lactate acid), poly(glycolic acid), polyorthoesters, polyanhydrides polyglycolic acid and polyglactin, and copolymers thereof. Other suitable biodegradable polymers include cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, zein, alginate, hyaluronic acid, mycelium, or copolymers or thereof.

In some embodiments, the polymer is permeable to gases, liquids, molecules in solution, or any combination thereof. In some embodiments, the polymer is impermeable to gases, liquids, molecules in solution, or any combination thereof.

In one embodiment, the polymer is at least one polymer bead. In another embodiment, the polymer is a substrate.

In various embodiments, the polymer is a biomimetic interface.

In some embodiments, the polymer is a film of a thickness of from about 0.01 nanometer to about 10 centimeters. For example, in some embodiments, the polymer is a film of a thickness of from about 0.1 nanometer to about 10 millimeters. In some embodiments, the polymer is a film of a thickness of from about 0.1 nanometer to about 10 micrometers. In some embodiments, the polymer is a film of a thickness of from about 0.1 nanometer to about 10 nanometers. In some embodiments, the polymer is a film of a thickness of from about 0.1 nanometer to about 1 millimeter. In some embodiments, the polymer is a film of a thickness of from about 1 nanometer to about 10 millimeters. In some embodiments, the polymer is a film of a thickness of from about 1 nanometer to about 1 micrometer. In some embodiments, the polymer is a film of a thickness of from about 5 nanometers to about 100 nanometers. In some embodiments, the polymer is a film of a thickness of from about 10 nanometers to about 50 nanometers.

In some embodiment, the composition can be of any configuration that allows for the contact of a mesogenic layer with the substrate. In various embodiments, the liquid crystal layer is placed on the substrate by electrospinning, spin coating, electrospraying, airbrushing, brushing, 3D printing, or any combination thereof of a liquid crystal on the substrate.

In various embodiments, the liquid crystal layer is placed on the substrate in a solvent-free matter. For example, in some embodiments, the liquid crystal layer is placed on the substrate by solvent-free electrospinning, solvent-free spin coating, solvent-free electrospraying, solvent-free airbrushing, solvent-free brushing, solvent-free 3D printing, or any combination thereof of a liquid crystal on the substrate.

In various embodiments, the liquid crystal layer is placed on the substrate in a polymer-free matter. For example, in some embodiments, the liquid crystal layer is placed on the substrate by polymer-free electrospinning, polymer-free spin coating, polymer-free electrospraying, polymer-free airbrushing, polymer-free brushing, polymer-free 3D printing, or any combination thereof of a liquid crystal on the substrate.

In various embodiments, the liquid crystal layer is placed on the substrate in a solvent-free and polymer-free matter. For example, in some embodiments, the liquid crystal layer is placed on the substrate by solvent-free and polymer-free electrospinning, solvent-free and polymer-free spin coating, solvent-free and polymer-free electrospraying, solvent-free and polymer-free airbrushing, solvent-free and polymer-free brushing, solvent-free and polymer-free 3D printing, or any combination thereof of a liquid crystal on the substrate.

In one embodiment, the substrate is chemically inert towards the mesogenic layer. In another embodiment, the substrate is reactive or interactive towards the mesogenic layer.

In various embodiments, the substrate comprises a cell tissue, organic layer, inorganic layer (e.g., metal, metal salt or metal oxide), or an organic-inorganic layer. For example, in some embodiments, the substrate is a skin, muscle, tissue layer, or any combination thereof.

In one embodiment, the substrate is a single layer substrate. In one embodiment, the substrate is a multilayer substrate. In one embodiment, the substrate comprises a uniform layer. In one embodiment, the substrate comprises a sub-layer. In some embodiment, the substrate is a stacked or side-by-side (i.e., adjacent) arrangement of substrate sublayers. In one embodiment, the substrate includes substrate sublayers that are arranged in a horizontally adjacent pattern. Thus, it should be understood that the substrate is not necessarily limited to single layers or sheets of materials. In addition, it should be understood that the surfaces of certain substrates, including the interface(s) of such substrate layers with other material(s) or layers(s), may be imperfect, wherein said surfaces represent an interpenetrating, entangled or convoluted network with other material(s) or layer(s). Similarly, it should also be understood that substrates may be uniform or discontinuous, such that the continuity of substrate layers along the length, width, and/or perimeter may be disturbed or otherwise interrupted by other layer(s) or material(s).

In one embodiment, the substrate is a rigid structure that is impermeable to liquids and gases. In this embodiment, the substrate consists of a glass plate onto which a metal, such as gold is layered by evaporative deposition. In one embodiment, the substrate is a glass plate (SiO₂) onto which a first metal layer, such as titanium, has been layered. A layer of a second metal, such as gold, can be then layered on top of the first metal layer.

In one embodiment, the substrate is permeable and it consists of a layer of gold, or gold over titanium, which is deposited on a polymeric membrane, or other material, that is permeable to liquids, vapors and/or gases. The liquids and gases can be pure compounds (e.g., chloroform, carbon monoxide) or they can be compounds that are dispersed in other molecules (e.g., aqueous protein solutions, herbicides in air, alcoholic solutions of small organic molecules). Useful permeable membranes include, but are not limited to, flexible cellulosic materials (e.g., regenerated cellulose dialysis membranes), rigid cellulosic materials (e.g., cellulose ester dialysis membranes), rigid polyvinylidene fluoride membranes, polydimethylsiloxane and track etched polycarbonate membranes.

In some embodiments, the nature of the surface of the substrate has a profound effect on the anchoring of the mesogenic layer that is associated with the surface. The surface can be engineered by the use of mechanical and/or chemical techniques. The surface of each of the above enumerated substrates can be substantially smooth. Alternatively, the surface can be roughened or patterned by rubbing, etching, grooving, stretching, stressing, impacting, nanoblasting, oblique deposition or other similar techniques known to those of skill in the art. Of particular relevance is the texture of the surface that is in contact with the mesogenic compounds.

In some embodiments, organic layers are utilized as substrate materials. In some embodiments, the organic layer is fabricated via thermal evaporation, ink-jet, organic vapor phase deposition (OVPD), organic vapor jet printing (OVJP), or any combination thereof. Other suitable fabrication methods of organic layers include spin coating and other solution-based processes. In some embodiments, the solution-based processes are carried out in nitrogen or an inert atmosphere.

In some embodiments, the substrate comprises an inorganic crystal, inorganic glass, or any combination thereof. For example, in some embodiments, the substrate is a glass, polymer, graphene, graphene oxide, graphite, metal, composite, wood, paper, rubber, fabric, fibrous network, mineral, brass, stones, natural stones used in watch dial manufacturing, laipis azul, meteorite, crystal, mineral, pearl, mother of pearl, artificial mineral (e.g., artificial sapphire), or any combination thereof. In addition, in some embodiments, the surface of the substrate is functionalized with a molecular layer, or with a polymer layer or layers, or any combination thereof.

In some embodiments, the substrate can be made of practically any physicochemically stable material. In one embodiment, the substrate material is non-reactive towards the constituents of the mesogenic layer. In some embodiments, the substrate is rigid or flexible. In some embodiments, the substrate is optically transparent or optically opaque. In some embodiments, the substrate is an electrical insulator, conductor, semiconductor, or any combination thereof. In some embodiments, the substrate can be either permeable or impermeable to materials, such as liquids, solutions, vapors and gases. In some embodiments, the substrate is substantially impermeable to liquids, vapors and/or gases or, alternatively, the substrates can be permeable to one or more of these classes of materials. Exemplary substrate materials include, but are not limited to, inorganic crystals, inorganic glasses, inorganic oxides, metals, organic polymers and combinations thereof.

In some embodiments, the substrate surface is prepared by rubbing, nanoblasting (i.e., abrasion of a surface with submicron particles to create roughness), or oblique deposition of a metal. In some embodiments, the substrate provides a uniform, homogenous surface, while in other embodiments, the surface is heterogenous. In some embodiments, the substrate is patterned.

In one embodiment, the substrate is glass or an organic polymer and the surface has been prepared by rubbing. Rubbing can be accomplished using virtually any material including tissues, paper, fabrics, brushes, polishing paste, etc. In one embodiment, the rubbing is accomplished by use of a diamond rubbing paste. In another embodiment, the face of the substrate that contacts the mesogenic compounds is a metal layer that has been obliquely deposited by evaporation.

In some embodiments, the substrate comprises an anisotropic surface prepared by nanoblasting a substrate with nanometer scale beads (e.g., about 1-200 nm, such as about 50-100 nm) at a defined angle of incidence (e.g., from about 5-85°, such as about 45°). The nanoblasted surface can be utilized as is or can be further modified, such as by obliquely depositing gold on the surface or by chemically functionalizing with a molecular layer so as to change its surface chemical and physical properties (Hohman J N et al., ACS Nano 2009, 3, 3, 527-536; Kim J et al., Nano Lett. 2014, 14, 5, 2946-2951; Schwartz J J et al., J. Am. Chem. Soc. 2016, 138, 18, 5957-5967).

In some embodiments, the substrate comprises an anisotropic surface prepared by stretching an appropriate substrate. For example, polymer substrates, such as polystyrene, can be stretched by heating to a temperature above the glass transition temperature of the substrate, applying a tensile force, and cooling to a temperature below the glass transition temperature before removing the force.

In some embodiments, the substrate comprises heterogenous features for use in the various devices and methods. In some embodiments, the heterogeneity is a uniform or non-uniform gradient in topography across the surface. For example, gold can be deposited onto a substrate at varying angles of incidence. Regions containing gold deposited at a near-normal angle of incidence will cause non-uniform anchoring of the liquid crystal, while areas in which the angle of incidence was greater than 10° will uniformly orient crystals. Alternatively, the heterogeneity may be the presence of two or more distinct scales topography distributed uniformly across the substrate.

In some embodiments, the substrate is patterned. The substrate can be patterned using techniques such as photolithography (Kleinfield et al., J. Neurosci. 8:4098-120 (1998)), photoetching, chemical etching, microcontact printing (Kumar et al., Langmuir 10:1498-511 (1994)), and chemical spotting.

The size and complexity of the pattern on the substrate is limited only by the resolution of the technique utilized and the purpose for which the pattern is intended. For example, using microcontact printing, features as small as 200 nm have been layered onto a substrate (e.g., Xia, Y.; Whitesides, G., J. Am. Chem. Soc. 117:3274-75 (1995)). Similarly, using photolithography, patterns with features as small as 1 &mgr;m have been produced (e.g., Hickman et al., J. Vac. Sci. Technol. 12:607-16 (1994); Smith R K et al., Progress in Surface Science, 2004, 75:1-68). Patterns which are useful in the present invention include those which comprise features such as wells, enclosures, partitions, recesses, inlets, outlets, channels, troughs, diffraction gratings and the like.

In some embodiments, the patterning is used to produce a substrate having a plurality of adjacent wells, wherein each of the wells is isolated from the other wells by a raised wall or partition and the wells do not fluidically communicate. Thus, a liquid crystal, or other substance, placed in a particular well remains substantially confined to that well. In some embodiments, the patterning allows the creation of channels through the device whereby a stimulus can enter and/or exit the device.

The pattern can be printed directly onto the substrate or, alternatively, a “lift off” technique can be utilized. In the lift off technique, a patterned resist is laid onto the substrate, an organic layer is laid down in those areas not covered by the resist and the resist is subsequently removed. Resists appropriate for use with the substrates of the present invention are known to those of skill in the art (e.g., Kleinfield et al., J. Neurosci. 8:4098-120 (1998); Liao W S et al., 2012, Science, 337:1517-1521). Following removal of the photoresist, a second organic layer, having a structure different from the first organic layer, can be bonded to the substrate on those areas initially covered by the resist. Using this technique, substrates with patterns having regions of different chemical characteristics can be produced. Thus, for example, a pattern having an array of adjacent wells can be created by varying the hydrophobicity/hydrophilicity, charge and other chemical characteristics of the pattern constituents. In one embodiment, hydrophilic compounds can be confined to individual wells by patterning walls using hydrophobic materials.

Similarly, positively or negatively charged compounds can be confined to wells having walls made of compounds with charges similar to those of the confined compounds. Similar substrate configurations are accessible through microprinting a layer with the desired characteristics directly onto the substrate (e.g., Mrkish, M.; Whitesides, G. M., Ann. Rev. Biophys. Biomol. Struct. 25:55-78 (1996); Liao W S et al, 2012, Science, 337:1517-1521).

In some embodiments, the patterned substrate controls the anchoring alignment of the liquid crystal. In one embodiment, the substrate is patterned with an organic compound (e.g., organic polymer) that forms a SAM. In one embodiment, the organic layer controls the azimuthal orientation and/or polar orientation of a supported mesogenic layer.

In addition to the ability of a substrate to anchor a mesogenic layer, an organic layer attached to the substrate is similarly able to provide such anchoring. A wide range of organic layers can be used in conjunction with the present invention. These include, but are not limited to, organic layers formed from organosulfur compounds (e.g., thiols and disulfides), organosilanes, amphiphilic molecules, cyclodextrins, polyols (e.g., poly(ethyleneglycol), poly(propylene glycol), fullerenes, and biomolecules.

An organic layer that is bound to, supported on or adsorbed onto, the surface of the substrate can anchor a mesogenic layer. As used herein, the term “anchoring” refers to the set of orientations adopted by the molecules in the mesogenic phase. The mesogenic layer will adopt particular orientations while minimizing the free energy of the interface between the organic layer and the mesogenic layer. The orientation of the mesogenic layer is referred to as an “anchoring direction.” A number of anchoring directions are possible.

In some embodiments, the particular anchoring direction adopted will depend upon the nature of the mesogenic layer, the organic layer and the substrate. Anchoring directions of use in the present invention include, for example, conical anchoring, degenerate anchoring, homeotropic anchoring, multistable anchoring, planar anchoring and tilted anchoring. In some embodiments, the anchoring is a planar anchoring or homeotropic anchoring.

The anchoring of mesogenic compounds by surfaces has been extensively studied for a large number of systems (e.g., Jerome, Rep. Prog. Phys. 54:391-451 (1991)). The anchoring of a mesogenic substance by a surface is specified, in general, by the orientation of the director of the bulk phase of the mesogenic layer. The orientation of the director, relative to the surface, is described by a polar angle (measured from the normal of the surface) and an azimuthal angle (measured in the plane of the surface).

Control of the anchoring of mesogens has been largely based on the use of organic surfaces prepared by coating surface-active molecules or polymer films on inorganic (e.g., silicon oxide) substrates followed by surface treatments, such as rubbing. Other systems which have been found useful include surfaces prepared through the reactions of organosilanes with various substrates (e.g., Yang et al., In Microchemistry: Spectroscopy and Chemistry in Small Domains; Masuhara et al., Eds.; North-Holland, Amsterdam, 1994; p.441).

Molecularly designed surfaces formed by organic layers on a substrate can be used to control both the azimuthal and polar orientations of a supported mesogenic layer. SAMs can be patterned on a surface. For example, patterned substrate or pattern organic layers made from CH₃(CH₂)₁₄SH and CH₃(CH₂)₁₅SH on obliquely deposited gold produce a supported mesogenic layer which is twisted 90°. Other anchoring modes are readily accessible by varying the chain length and the number of species of the organic layer constituents (e.g., Gupta and Abbott, Science 276:1533-1536 (1997)).

Transitions between anchoring modes have been obtained on a series of organic layers by varying the structure of the organic layer. The structural features which have been found to affect the anchoring of mesogenic compounds include, for example, the density of molecules within the organic layer, the size and shape of the molecules constituting the organic layer and the number of individual layers making up the bulk organic layer.

The density of the organic layer on the substrate has been shown to have an effect on the mode of mesogen anchoring. For example, transitions between homeotropic and degenerate anchorings have been obtained on surfactant monolayers by varying the density of the monolayers (e.g., Proust et al., Solid State Commun. 11: 1227-30 (1972)). Thus, it is within the scope of the present invention to tailor the anchoring mode of a mesogen by controlling the density of the organic layer on the substrate.

The molecular structure, size and shape of the individual molecules making up the organic layer also affects the anchoring mode. For example, it has been demonstrated that varying the length of the aliphatic chains of surfactants on a substrate can also induce anchoring transitions; with long chains, a homeotropic anchoring is obtained while with short chains, a conical anchoring is obtained with the tilt angle O increasing as the chain becomes shorter (e.g., Porte, J. Physique 37:1245-52 (1976)). Additionally, recent reports have demonstrated that the polar angle of the mesogenic phase can be controlled by the choice of the constituents of the organic layer. e.g., Gupta and Abbott, Langmuir 12:2587-2593 (1996). The anchor can also include switchable elements, such as the photoswitchable chemical moiety azobenzene, so that the anchor can change between two or more states (e.g., Abendroth et al., ACS Nano 9:7746-7768 (2015)). The stimulation to induce switching could be light, electrochemical potential, electric field, pH, chemistry, and mechanical motion, among others. Thus, it is within the scope of the present invention to engineer the magnitude of the anchoring shift as well as the type of shift by the judicious choice of organic layer constituents.

A wide variety of organic layers are useful in practicing the present invention. These organic layers can comprise monolayers, bilayers and multilayers. Furthermore, the organic layers can be attached by covalent bonds, ionic bonds, physisorption, chemisorption and the like, including, but not limited to, hydrophobic interactions, hydrophilic interactions, van der Waals interactions and the like.

A wide variety of reaction types are available for the functionalization of a substrate surface. For example, substrates constructed of a polymer, such as polypropylene, can be surface derivatized by chromic acid oxidation, and subsequently converted to hydroxylated or aminomethylated surfaces. Substrates made from highly crosslinked divinylbenzene can be surface derivatized by chloromethylation and subsequent functional group manipulation. Additionally, functionalized substrates can be made from etched, reduced polytetrafluoroethylene.

When the substrates are constructed of a siliceous material, such as glass, the surface can be derivatized by reacting the surface Si—OH, SiO—H, and/or Si—Si groups with a functionalizing reagent. When the substrate is made of a metal film, the surface can be derivatized with a material displaying avidity for that metal.

Substrates can be made reactive by plasma oxidation or by other means of chemical oxidation.

The hydrophilicity of the substrate surface can be enhanced by reaction with polar molecules such as amine-, hydroxyl- and polyhydroxylcontaining molecules. Representative examples include, but are not limited to, polylysine, polyethyleneimine, poly(ethylene glycol) and poly(propylene glycol). Suitable functionalization chemistries and strategies for these compounds are known in the art (e.g., Dunn, R. L., et al., Eds. Polymeric Drugs and Drug Delivery Systems, ACS Symposium Series Vol. 469, American Chemical Society, Washington, D.C. 1991).

The hydrophobicity of the substrate surface can be modulated by using a hydrophobic spacer arm, such as, for example, long chain diamines, long chain thiols, amino acids, etc. Representative hydrophobic spacers include, but are not limited to, 1,6-hexanediamine, 1,8-octanediamine, 6-aminohexanoic acid and 8-aminooctanoic acid.

The substrate surface can also be made surface-active by attaching to the substrate surface a spacer that has surfactant properties. Compounds useful for this purpose include, for example, aminated or hydroxylated detergent molecules such as, for example, 1-aminododecanoic acid.

In various embodiments, the composition further comprises a “spacer”. In some embodiments, the “spacer” is a graphene, graphite, graphene oxide, boron nitride, or any combination thereof. In some embodiments, the composition comprises a “spacer” between the liquid crystal layer and at least a portion of the surface of the substrate. In some embodiments, the “spacer” acts as a glue to hold the liquid crystal layer and at least a portion of the surface of the substrate. For example, in some embodiments, the composition comprises graphene, graphite, graphene oxide, or any combination thereof that acts as a glue to hold the liquid crystal layer and at least a portion of the surface of the substrate.

In one embodiment, the liquid crystal is a liquid crystal scaffold. Thus, in one embodiment, the liquid crystal scaffold is 100% w/v liquid crystal.

In one embodiment, the composition comprises a liquid crystal scaffold. In one embodiment, the composition comprises a nonwoven liquid crystal scaffold. In one embodiment, the composition comprises an electospun nonwoven liquid crystal scaffold. For example, in one embodiment, the composition is an electrospun nonwoven cholesteryl ester liquid crystal scaffold.

In some embodiments, the electrospun liquid crystal scaffold has a uniform depth. In some embodiments, the electrospun liquid crystal scaffold has a non-uniform depth.

In some embodiments, the electrospun liquid crystal scaffold is functionalized, for example, by the addition of passive or active agents, such as additional therapeutic or biological agents.

In some embodiments, the electrospun liquid crystal scaffold comprises electrospun fibers. There are many factors involved in the electrospinning process, which may affect scaffolds fiber diameter and pore size. Examples of such variables include, but are not limited to, solution viscosity, surface tension, and viscoelasticity of the spinning solution. These are directly related to the concentration of, and molecular weight of the polymer, as well as the solvent used. The dielectric properties of the solution also play a key role (Kowalczyk et al., Biomacromolecules. 2008 Jul;9(7):2087-90; Thompson et al., J. Polymer. 2007;48:6913-6922; Mitchell and Sanders, J Biomed Mater Res A. 2006 Jul;78(1): 1 10-20).

In some embodiments, the electrospun liquid crystal scaffold comprises a polymer. In one embodiment, the electrospun fibers comprise a polymer. For example, in one embodiment, the electrospun fibers comprise an electrospun polymer. In one embodiment, the electrospun fibers comprise an electrospun polycaprolactone or a derivative thereof.

In some embodiments, the electrospun liquid crystal scaffold comprises a polylactide or a derivative thereof. In some embodiments, the electrospun liquid crystal scaffold comprises polyurethane, preferably polyurethane based on hexamethylenediamine, polylactide derivatives, and chitosan derived material.

In some embodiments, the electrospun liquid crystal scaffold comprises a combination of synthetic polymers and naturally occurring biological material, for example a combination of collagen and PLGA. The relative amounts of the synthetic polymers and naturally occurring biological material in the matrix can be tailored to specific applications.

In one embodiment, the electrospun liquid crystal scaffolds may comprise a co-polymer. For example, in one embodiment, the electrospun fibers may comprise a co-polymer. The term “co-polymer” as used herein is intended to encompass co-polymers, ter-polymers, and higher order multiple polymer compositions formed by block, graph or random combination of polymeric components. Examples of such co-polymers include, but are not limited to poly(L-lactic-co-caprolactone), poly(ethylene glycol-co-lactide), poly(D,L-lactide-co-glycolide), poly(ethylene-co-vinyl alcohol), poly(D,L-lactic-co-glycolic acid) and PLGA-B-PEG-NH2, poly(D,L-lactic-co-glycolide), collagen and elastin, poly(L-lactic-co-caprolactone), collagen, poly(L-lactic acid), hydroxylapitate, poly(lactic-co-glycolic acid), and any combination thereof.

In some embodiments, the polymer and/or co-polymer are electrospun onto a template. In some embodiments, the template comprises a conductive collector having a pattern thereon. The collector may be formed of any electrically conductive material, such as a metal. In some embodiments, the collector is formed from aluminum, such electroplated aluminum or an aluminum sheet, such as aluminum foil or formed from an electrically conductive material comprising aluminum, brass, copper, steel, tin, nickel, titanium, silver, gold or platinum.

The pattern may be formed on the collector using any suitable method known in the art. In one embodiment, the pattern may be microfabricated on a surface of the collector. By way of example, the pattern may be microfabricated using microlithography, bonding, etching or injection molding. In one embodiment the pattern may be created by photolithography, microstereolithography or shadow masking. Preferably, the microfabricated three dimensional structures are microfabricated using microstereolithography, more preferably by a layer by layer photocuring approach based on the patterning of photocurable polymers, for example polyethylene glycol diacrylate.

In some embodiments, the pattern is non-conductive/insulating. Examples of non-conductive/insulating polymers, from which the pattern may be formed include example acrylated polymers, such as polyethylene glycol diacrylate, polyethylene glycol dimethacrylate or pentaerythritol tetraacrylate. Alternatively, the pattern may be formed from thiol-ene based polymers, or ceramics, such as ORMOCER.

In some embodiments, the pattern is dimensioned to provide a scaffold comprising at least one cavity capable of acting as a stem cell niche. Preferably, the pattern provides a scaffold having a cavity having a diameter of from 10 μm to 500 μm, preferably from 50 μm to 400 μm, still more preferably from 150 μm to 300 μm and a depth of from 10 μm to 1000 μm, preferably a depth of from 50 μm to 150 μm.

In some embodiments, the pattern is dimensioned to provide a scaffold of nonuniform depth.

In some embodiments, the pattern is dimensioned to provide a scaffold comprising multiple cavities.

In some embodiments, the electrospun liquid crystal scaffold comprises at least one cavity. For example, in some embodiments, the electrospun liquid crystal scaffold comprises at least one cavity therein capable of acting as a stem cell niche. In some embodiments, the electrospun liquid crystal scaffold comprises an edible polymer or co-polymer, wherein said scaffold comprises at least one cavity therein capable of acting as a stem cell niche.

In some embodiments, the cavity has a diameter of from 10 μm to 500 μm, preferably from 50 μm to 400 μm, still more preferably from 150 μm to 300 μm and a depth of from 10 μm to 1000 μm, preferably from 50 μm to 150 μm. Preferably, the scaffold comprises multiple cavities, for example at least 5, 10 15, 20, 50, 100, 200 or 500 cavities.

Thus, in some embodiments, the cavity comprises at least one cell. Examples of such cell include, but are not limited to a tissue cell, epithelial cell, endothelial cell, muscle cell, neuron, adipocyte, stem cell (e.g., a muscle stem cell, mesenchymal stem cell, epithelial stem cell, etc.), or any combination thereof.

The aforementioned cells may be seeded into the cavity by any technique known in the art. For example, in some embodiments, the cells may be electrosprayed into the cavity, pipetted into the cavity, flowed into the cavity via a bioreactor, or any combination thereof.

In various aspects of the present invention, the composition may further comprise at least one cell that promotes maintenance of the stem cell, for example, a specialized support cell for the muscle cell, such as fibroblasts.

In some embodiments, the composition may further comprise any extracellular matrix component, such as fibronectin, vitronectin, collagen, laminin.

In some embodiments, the composition may further comprise any circulating materials involved in wound healing, such as fibrin (formed during clot formation and a natural adhesive) and heparin (secreted during wound healing and able to bind and immobilize short-lived growth factors, which are subsequently slowly released to promote local cell migration and proliferation). The cavity may also comprise growth factors and/or short protein fragments.

In some embodiments, the composition may further comprise naturally occurring materials. Examples of suitable naturally occurring materials include, but are not limited to, amino acids, polypeptides, denatured peptides such as gelatin from denatured collagen, carbohydrates, lipids, nucleic acids, glycoproteins, lipoproteins, glycolipids, glycosaminoglycans, and proteoglycans. In one embodiment the naturally occurring material is an extracellular matrix material, for example collagen, fibrin, elastin, laminin, fibronectin, heparin, fibrinogen. Such extracellular matrix material may be isolated from cells, such as mammalian cells, for example of human origin. Preferably the naturally occurring material is collagen. Alternatively, the naturally occurring polymer is chitin. Preferably the scaffold is biodegradable, i.e. is formed of biodegradable materials, such as biodegradable polymers naturally occurring biological material. Examples of suitable biodegradable materials include, but are not limited to collagen, poly(alpha esters) such as poly(lactate acid), poly(glycolic acid), polyorthoesters, polyanhydrides polyglycolic acid and polyglactin, and copolymers thereof.

For example, in some embodiments, the properties of the electrospun liquid crystal scaffolds can be adjusted in accordance with the needs and specifications of the cells to be suspended and grown within them. The porosity, for instance, can be varied in accordance with the method of making the electrospun materials matrix. As used herein, a natural biological polymer material can be a naturally occurring organic material including any material naturally found in the body of a mammal, plant, or other organism.

In various aspects of the present invention, the liquid crystal (i.e., 100% w/v liquid crystal) or the composition thereof for inducing cell-culturing and/or generating at least one cell tissue layer induce cell tissue generation or regeneration.

In other aspects of the present invention, the liquid crystal (i.e., 100% w/v liquid crystal) or the composition thereof for inducing cell-culturing and/or generating at least one cell tissue layer exercise the cultured cells through treadmill action. In some embodiments, the liquid crystal or the composition thereof exercise the cultured cells through treadmill action, with or without external stimulation. This action has important advantages in both cellular agriculture and regenerative medicine.

In other aspects of the present invention, the liquid crystal (i.e., 100% w/v liquid crystal) or the composition thereof for inducing cell-culturing and/or generating at least one cell tissue layer induce muscle tissue regeneration.

In other aspects of the present invention, the liquid crystal (i.e., 100% w/v liquid crystal) or the composition thereof for inducing cell-culturing and/or generating at least one cell tissue layer induce muscle repair.

In various aspects of the present invention, the liquid crystal (i.e., 100% w/v liquid crystal) or the composition thereof for inducing cell-culturing and/or generating at least one cell tissue layer induce wound-healing.

Thus, in some aspects of the invention, the composition is a pharmaceutical composition.

In some embodiments, the composition further comprises one or more therapeutic agents.

In one embodiment, the therapeutic agent is a hydrophobic therapeutic agent. In one embodiment, the therapeutic agent is a hydrophilic therapeutic agent. Examples of such therapeutic agents include, but are not limited to, one or more drugs, proteins, amino acids, peptides, antibodies, antibiotics, anti-inflammatory agents, anti-infection agents, anti-bacterial agents, anti-viral agents, anti-fungal agents, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

In one embodiment, the therapeutic agent may be an anti-infection agent. Any suitable anti-infection agent may be used in the compositions and methods of the present disclosure. The selection of a suitable anti-infection agent may depend upon, among other things, the type of infection to be treated and the composite of the present disclosure. In certain embodiments, the anti-infection agent may be an anti-bacterial agent, anti-fungal agent, anti-viral agent, or any combination thereof. Thus, in certain embodiments, the anti-infection agent may be effective for treating one or more of bacterial infection, viral infection, fungal infection, or any combination thereof.

Examples of antibacterial agents or antibiotics include, but are not limited to, aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), macrolides (e.g., azithromycin, carbomycin, clarithomycin, dirithromycin, erythromycin, and erythromycin acistrate), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, ciprofloxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

Additional non-limiting examples of antibacterial agents include Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmnenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Gatifloxacin Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafingin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Suifabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin.

Examples of anti-fungal agent include, but are not limited to, polyenes (e.g., amphotericin b, candicidin, mepartricin, natamycin, and nystatin), allylamines (e.g., butenafine, and naftifine), imidazoles (e.g., bifonazole, butoconazole, chlordantoin, flutrimazole, isoconazole, ketoconazole, and lanoconazole), thiocarbamates (e.g., tolciclate, tolindate, and tolnaftate), triazoles (e.g., fluconazole, itraconazole, saperconazole, and terconazole), bromosalicylchloranilide, buclosamide, calcium propionate, chlorphenesin, ciclopirox, azaserine, griseofulvin, oligomycins, neomycin undecylenate, pyrrolnitrin, siccanin, tubercidin, and viridin. Additional examples of antifungal compounds include but are not limited to Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofingin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate; Tolindate; Tolnaftate; Triacetin; Triafuigin; Undecylenic Acid; Viridoflilvin; Zinc Undecylenate; and Zinoconazole Hydrochloride.

Examples of anti-viral agents include, but are not limited to, proteins, polypeptides, peptides, fusion protein antibodies, nucleic acid molecules, organic molecules, inorganic molecules, and small molecules that inhibit or reduce the attachment of a virus to its receptor, the internalization of a virus into a cell, the replication of a virus, or release of virus from a cell. Many examples of antiviral compounds that can be used in combination with the compounds of the invention are known in the art and include but are not limited to: rifampicin, nucleoside reverse transcriptase inhibitors (e.g., AZT, ddl, ddC, 3TC, d4T), non-nucleoside reverse transcriptase inhibitors (e.g., Efavirenz, Nevirapine), protease inhibitors (e.g., aprenavir, indinavir, ritonavir, and saquinavir), idoxuridine, cidofovir, acyclovir, ganciclovir, zanamivir, amantadine, and Palivizumab. Other examples of anti-viral agents include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime, zinc, heparin, anionic polymers.

In one embodiment, the therapeutic agent may be an anti-inflammatory agent. Any suitable anti-inflammatory agent may be used in the compositions and methods of the present disclosure. The selection of a suitable anti-inflammatory agent may depend upon, among other things, the type of inflammation to be treated and the composite of the present disclosure. Examples of anti-inflammatory agents include, but are not limited to, non-steroidal anti-inflammatory drugs (NSAIDs), steroidal anti-inflammatory drugs, beta-agonists, anticholingeric agents, antihistamines (e.g., ethanolamines, ethylenediamines, piperazines, and phenothiazine), and methyl xanthines. Examples of NSAIDs include, but are not limited to, aspirin, ibuprofen, salicylates, acetominophen, celecoxib, diclofenac, etodolac, fenoprofen, indomethacin, ketoralac, oxaprozin, nabumentone, sulindac, tolmentin, rofecoxib, naproxen, ketoprofen and nabumetone. Such NSAIDs function by inhibiting a cyclooxgenase enzyme (e.g., COX-1 and/or COX-2). Examples of steroidal anti-inflammatory drugs include, but are not limited to, glucocorticoids, dexamethasone, cortisone, hydrocortisone, prednisone, prednisolone, triamcinolone, azulfidine, and eicosanoids such as prostaglandins, thromboxanes, and leukotrienes.

### Methods of Preparation

The present invention also relates, in part, to methods, techniques, and strategies for fabricating and characterizing the liquid crystals or compositions thereof described herein. In one aspect, the present invention relates, in part, to methods of generating the liquid crystal described herein. In another aspect, the present invention relates, in part, to methods generating the liquid crystal scaffold described herein.

In some embodiments, the method is a solvent-free method, polymer-free method, or a combination thereof.

For example, in one embodiment, the method of generating a liquid crystal (e.g., liquid crystal scaffold comprising 100% w/v liquid crystal) comprise generating a liquid crystal mesogen comprising cholesteryl oleyl carbonate or a derivative or a salt thereof, cholesteryl pelargonate or a derivative or a salt thereof, cholesteryl benzoate or a derivative or a salt thereof, or any combination thereof; melting the liquid crystal mesogen; and cooling the liquid crystal mesogen to generate a viscous liquid.

In various embodiments, the liquid crystal mesogen can be generated using any method described herein.

In some embodiments, the liquid crystal composition can be generated using any method described herein. In one embodiment, the method of generating a liquid crystal composition (e.g., a liquid crystal scaffold) comprise generating a liquid crystal mesogen comprising cholesteryl oleyl carbonate or a derivative or a salt thereof, cholesteryl pelargonate or a derivative or a salt thereof, cholesteryl benzoate or a derivative or a salt thereof, or any combination thereof; melting the liquid crystal mesogen; cooling the liquid crystal mesogen to generate a viscous liquid; and placing the viscous liquid on at least a portion of a surface of a first substrate to generate the liquid crystal scaffold. For example, in some embodiments, the liquid crystal scaffold can be generated by melting the liquid crystal mesogen at about 60° C.; cooling the liquid crystal mesogen to generate a viscous liquid; and placing the viscous liquid on at least a portion of the internal surface of the substrate.

In some embodiments, the viscous liquid can be placed on at least a portion of the internal surface of the substrate using any method described herein. For example, in one embodiment, the viscous liquid can be placed on at least a portion of the internal surface of the substrate using solvent-free electrospinning, solvent-free spin coating, solvent-free electrospraying, solvent-free airbrushing, solvent-free brushing, or any combination thereof.

In various embodiments, the substrate can be prepared using any method described herein. For example, in some embodiments, the substrate surface is prepared by rubbing, nanoblasting (i.e., abrasion of a surface with submicron particles to create roughness), or oblique deposition of a metal. In some embodiments, the substrate provides a uniform, homogenous surface, while in other embodiments, the surface is heterogenous. In some embodiments, the substrate is patterned.

In one embodiment, the method comprises a polymer. In one embodiment, the method of generating a liquid crystal composition (e.g., a liquid crystal scaffold) comprise generating a liquid crystal mesogen comprising cholesteryl oleyl carbonate or a derivative or a salt thereof, cholesteryl pelargonate or a derivative or a salt thereof, cholesteryl benzoate or a derivative or a salt thereof, or any combination thereof; melting the liquid crystal mesogen; cooling the liquid crystal mesogen to generate a viscous liquid; and mixing the viscous liquid with at least one polymer to generate the liquid crystal scaffold.

In other embodiments, the present invention provides a method for producing an electrospun scaffold, comprising electrospinning a polymer or co-polymer onto a template comprising a conductive collector having a three-dimensional pattern thereon, wherein said electrospun polymer or copolymer preferentially deposits onto said three-dimensional pattern.

In other embodiments, the method comprises a solvent. In some embodiments, the solvent serves as a medium for a reaction that generates a liquid crystal mesogen comprising cholesteryl oleyl carbonate or a derivative or a salt thereof, cholesteryl pelargonate or a derivative or a salt thereof, cholesteryl benzoate or a derivative or a salt thereof, or any combination thereof. In some embodiments, the solvent serves as a medium for a medium for the distribution of components of different phases or extraction of components into said solvent. For example, in one embodiment, the solvent serves as a medium for a medium for the distribution of the viscous liquid crystals liquid while placing the viscous liquid on at least a portion of a surface of a first substrate to generate the liquid crystal layer on the substrate.

For example, in one embodiment, the method of generating a liquid crystal composition (e.g., a liquid crystal scaffold) comprise generating a liquid crystal mesogen comprising cholesteryl oleyl carbonate or a derivative or a salt thereof, cholesteryl pelargonate or a derivative or a salt thereof, cholesteryl benzoate or a derivative or a salt thereof, or any combination thereof; melting the liquid crystal mesogen; cooling the liquid crystal mesogen to generate a viscous liquid; dispersing the viscous liquid and at least one polymer in a solvent; and electrospinning the liquid crystal and the at least one polymer to generate the liquid crystal scaffold.

### Methods of Use

The present invention also provides a method of inducing cell growth, cell-culturing, generating at least one cell tissue layer, or any combination thereof using at least one liquid crystal of the present invention (i.e., 100% w/v liquid crystal). In another aspect, the present invention provides a method of inducing cell-culturing, generating at least one cell tissue layer, or a combination thereof using at least one liquid crystal composition of the present invention (i.e., less than 100% w/v liquid crystal).

In various embodiments, the cell is any cell described herein. For example, in one embodiment, the cell is a mammalian cell. In other embodiments, the cell is an avian muscle cell, such chicken muscle cell, duck muscle cell, turkey muscle cell, quail muscle cell, etc., avian fat cell, such chicken fat cell, duck fat cell, turkey fat cell, quail fat cell, etc., bovine muscle cell, bovine fat cell, swine/porcine muscle cell, swine/porcine fat cell, sheep muscle cell, sheep fat cell, goat muscle cell, goat fat cell, piscine muscle cell, such as tuna muscle cell, salmon muscle cell, snapper muscle cell, cod muscle cell, etc., piscine fat cell, such as tuna fat cell, salmon fat cell, snapper fat cell, cod fat cell, etc., shellfish muscle cell, such as lobster muscle cell, crab muscle cell, shrimp muscle cell, crayfish muscle cell, clam muscle cell, oyster muscle cell, mussel muscle cell, etc., or any combination thereof.

In various aspects, the present invention provides a method of inducing cell-culturing of an avian muscle cell, such chicken muscle cell, duck muscle cell, turkey muscle cell, quail muscle cell, etc., avian fat cell, such chicken fat cell, duck fat cell, turkey fat cell, quail fat cell, etc., bovine muscle cell, bovine fat cell, swine/porcine muscle cell, swine/porcine fat cell, sheep muscle cell, sheep fat cell, goat muscle cell, goat fat cell, piscine muscle cell, such as tuna muscle cell, salmon muscle cell, snapper muscle cell, cod muscle cell, etc., piscine fat cell, such as tuna fat cell, salmon fat cell, snapper fat cell, cod fat cell, etc., shellfish muscle cell, such as lobster muscle cell, crab muscle cell, shrimp muscle cell, crayfish muscle cell, clam muscle cell, oyster muscle cell, mussel muscle cell, etc., or any combination thereof.

In other aspects, the present invention provides a method of generating at least one avian muscle tissue layer, such chicken muscle tissue layer, duck muscle tissue layer, turkey muscle tissue layer, quail muscle tissue layer, etc., avian fat tissue layer, such chicken fat tissue layer, duck fat tissue layer, turkey fat tissue layer, quail fat tissue layer, etc., bovine muscle tissue layer, bovine fat tissue layer, swine/porcine muscle tissue layer, swine/porcine fat tissue layer, sheep muscle tissue layer, sheep fat tissue layer, goat muscle tissue layer, goat fat tissue layer, piscine muscle tissue layer, such as tuna muscle tissue layer, salmon muscle tissue layer, snapper muscle tissue layer, cod muscle tissue layer, etc., piscine fat tissue layer, such as tuna fat tissue layer, salmon fat tissue layer, snapper fat tissue layer, cod fat tissue layer, etc., shellfish muscle tissue layer, such as lobster muscle tissue layer, crab muscle tissue layer, shrimp muscle tissue layer, crayfish muscle tissue layer, clam muscle tissue layer, oyster muscle tissue layer, mussel muscle tissue layer, etc., or any combination thereof. Thus, in various embodiments, the present invention provides a method of generating a synthetic meat (e.g., avian meat, bovine meat, fish meat, shellfish meat, goat meat, swine meat, sheep meat, etc.).

In another aspect, the present invention provides a method of inducing and/or propagating cellular agriculture of at least one cell or cell tissue layer of interest (e.g., human organ, cancer cell models, cancer spheroids for drug testing, etc.). For example, in one embodiment, the method induces and/or propagates organ cell tissue layer of interest. Thus, in one aspect, the present invention provides a method of generating organoids mimicking organs and/or multiple organs.

In another aspect, the present invention provides a method of inducing wound-healing in a subject in need thereof. In some embodiments, the method comprises inducing wound-healing of burns.

In another aspect, the present invention provides a method of inducing skin grafting in a subject in need thereof.

In another aspect, the present invention provides a method of inducing muscle tissue generation or regeneration.

In another aspect, the present invention provides a method of inducing muscle repair.

In another aspect, the present invention provides a method of exercising at least one cultured cell on a liquid crystal or a composition thereof through a treadmill action. In one embodiment, the method comprises a stimulus. In one embodiment, the stimulus is any stimulus described herein. For example, in some embodiments, the stimulus is selected from the group consisting of a light stimulus, electrical stimulus, and mechanical stimulus.

In various embodiments, the method comprises administering at least one liquid crystal of the present invention to the subject in need thereof. In various embodiments, the method comprises administering at least one composition of the present invention to the subject in need thereof. In one embodiment, the method comprises applying the at least one liquid crystal or the composition thereof to the subject in need thereof. For example, in one embodiment, the method comprises applying the at least one liquid crystal or the composition thereof to a skin of the subject in need thereof to induce cell growth of skin and/or muscle cells and promote wound-healing.

In various embodiments, the method comprises a stimulus. In various embodiments, the stimulus induces cell growth, cell-culturing, generating at least one cell tissue layer, wound healing, or any combination thereof. For example, in one embodiment, the method comprises thermal stimulus. For example, in one embodiment, the temperature of between about 36° C. to about 40° C. promotes the formation of liquid crystal striations inducing cell growth, cell-culturing, generating at least one cell tissue layer, wound healing, and any combination thereof.

In another aspect, the present invention provides a method of improving health of a subject in need thereof. For example, in one embodiment, the present invention provides a method of improving skin health of a subject in need thereof. Thus, in one embodiment, the present invention provides a method of improving skin health of a subject in need thereof, which prevents or treats skin cancer.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Cholesteryl Ester Liquid Crystal (CLC) Nanofibers for Tissue Engineering Applications

Liquid-crystal-based biomaterials provide promising platforms for the development of dynamic and responsive interfaces for tissue engineering. Cholesteryl ester liquid crystals (CLCs) are particularly well suited for these applications, due to their roles in cellular homeostasis and their intrinsic ability to organize into supramolecular helicoidal structures on the mesoscale. However, to date, CLCs have been a largely overlooked class of mesogens, even though they play key roles in modulating/stabilizing cell membranes, maintaining cellular homeostasis, and regulating signaling processes. These properties make CLCs attractive candidates for tissue engineering applications (Maxfield F R et al., 2005, Nature, 438:612-621). Previous studies showed that poly(lactic acid) scaffolds with covalently bound cholesterol moieties promoted the adhesion, migration, and proliferation of 3T3 fibroblasts (Hwang J J et al., 2002, Proc. Natl. Acad. Sci. USA, 99:9662-9667). Films of LC-based elastomers and networks have also been used to form three dimensional (3D) porous scaffolds that promoted the 10 proliferation and function of muscle and cardiac cells (Gao Yet al., 2016, ACS Macro Lett., 5:4-9; Ferrantini C et al., 2019, Circ. Res., 124:e44—e54; Martella D et al., 2019, Adv. Healthc. Mater., 8:1801489).

In one study, mouse myoblast cells (C2C12s) encapsulated within injectable peptide amphiphile liquid crystal scaffolds maintained their proliferation, differentiation, and maturation potentials in both in vitro and in vivo (murine) models (Sleep E et al., 2017, Proc. Natl. Acad. Sci. USA, 114:E7919—E7928). Previous studies have also demonstrated that topological defects, such as disinclinations, induced the formation of human fibroblast monolayers in fibronectin-free cell culture conditions (Turiv T et al., 2020, Sci. Adv., 6:eaaz6485). However, these LC-based scaffolds were nanometer-thin samples tethered to glass, which made them unsuitable for biomedical implantation.

The present studies describe the development of a nonwoven CLC electrospun scaffold by dispersing three cholesteryl ester-based mesogens within polycaprolactone (PCL). The ratio of the mesogens was judiciously tuned so that the CLC was in the mesophase at the cell culture incubator temperature of 37° C. In these scaffolds, the PCL polymer provided an elastic bulk matrix while the homogenously dispersed CLC established a viscoelastic fluid-like interface. Atomic force microscopy (AFM) revealed that the 50% (w/v) CLC-S exhibited a mesophase with topographic striations typical of liquid crystals. Additionally, the CLC-S favorable wettability and ultra-soft fiber mechanics enhanced cellular attachment and proliferation. Increasing the CLC concentration within the composite, enhanced myoblast adhesion strength, shortened differentiation times in culture, and promoted myofibril formation in vitro with mouse myoblast cell lines.

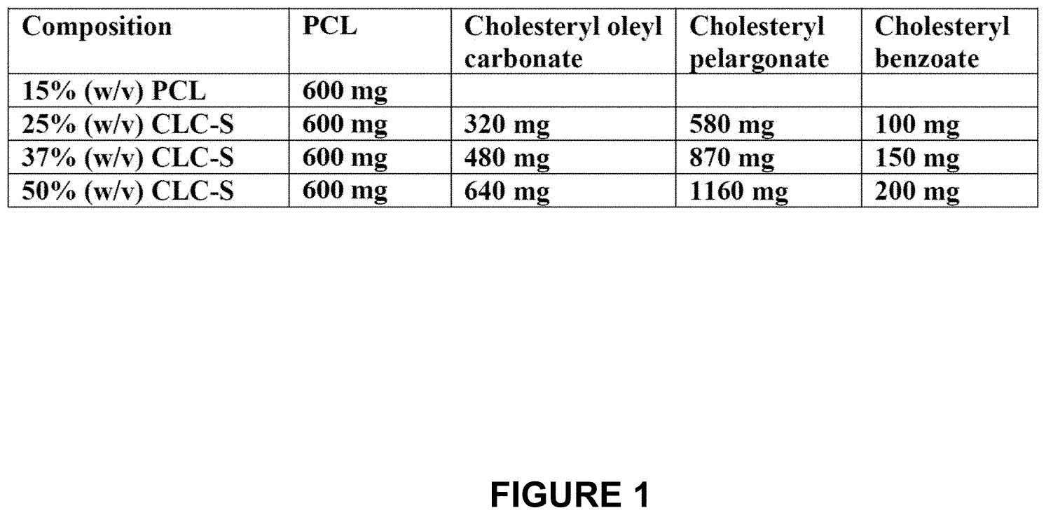

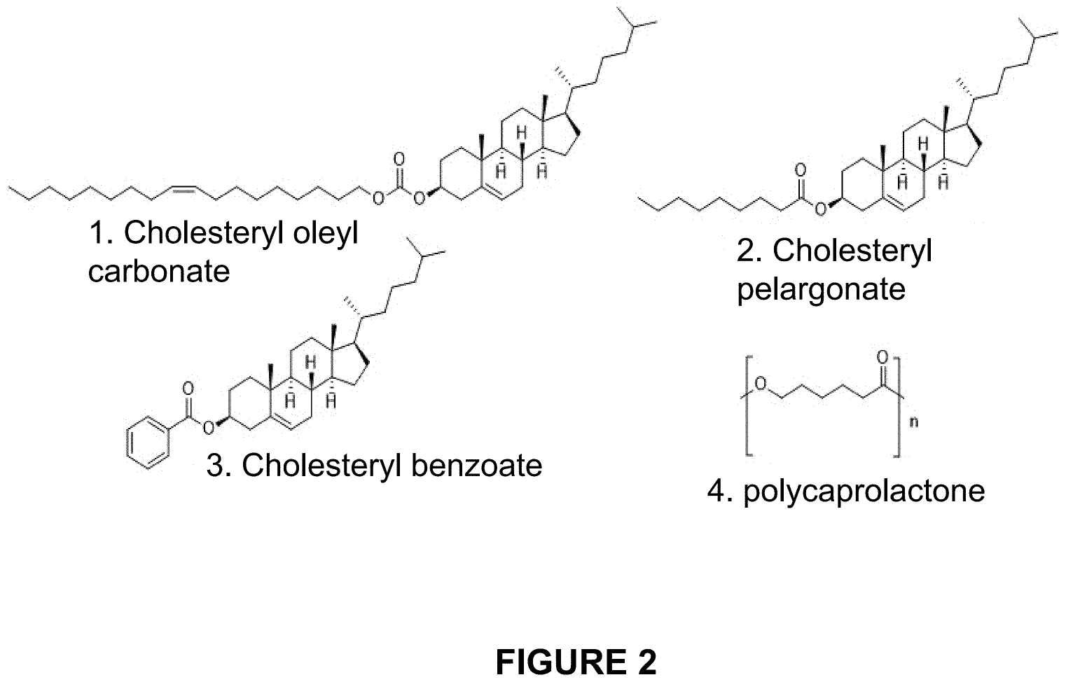

In this study, a nonwoven CLC-S was electrospun by simply dispersing cholesteryl oleyl carbonate, cholesteryl pelargonate, and cholesteryl benzoate in a PCL solvent while stirring. By using a reported ratio of the cis-unsaturated fatty acid cholesteryl oleyl carbonate to the saturated fatty acid cholesteryl pelargonate found in FIG. 1 and FIG. 2, the CLC was engineered to have its mesophase occur at 36-40° C., which coincided with the temperature of the cell culture incubator; the melting temperature of the liquid crystal mixture increased with the percent composition of cholesteryl pelargonate due to the more efficient packing of saturated fatty acids (Van Hecke R G et al., 2005, J. Chem. Educ., 82:1349-1354). Although not bound by any particular theory, it was hypothesized that the incorporation of the CLC phase generated a biomimetic interface that responded to cellular mechanical cues. To test this, mouse C2C12 myoblasts were cultured on the CLC-S scaffolds and their growth and development were characterized via immunohistochemistry, scanning electron microscopy (SEM), and AFM.

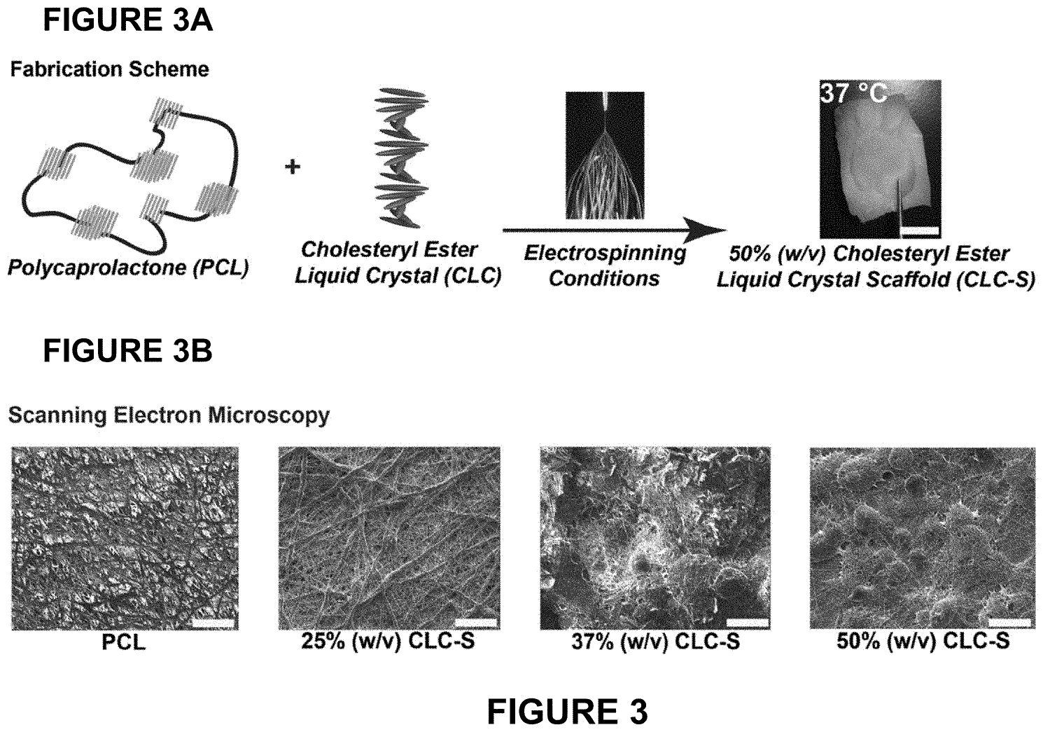

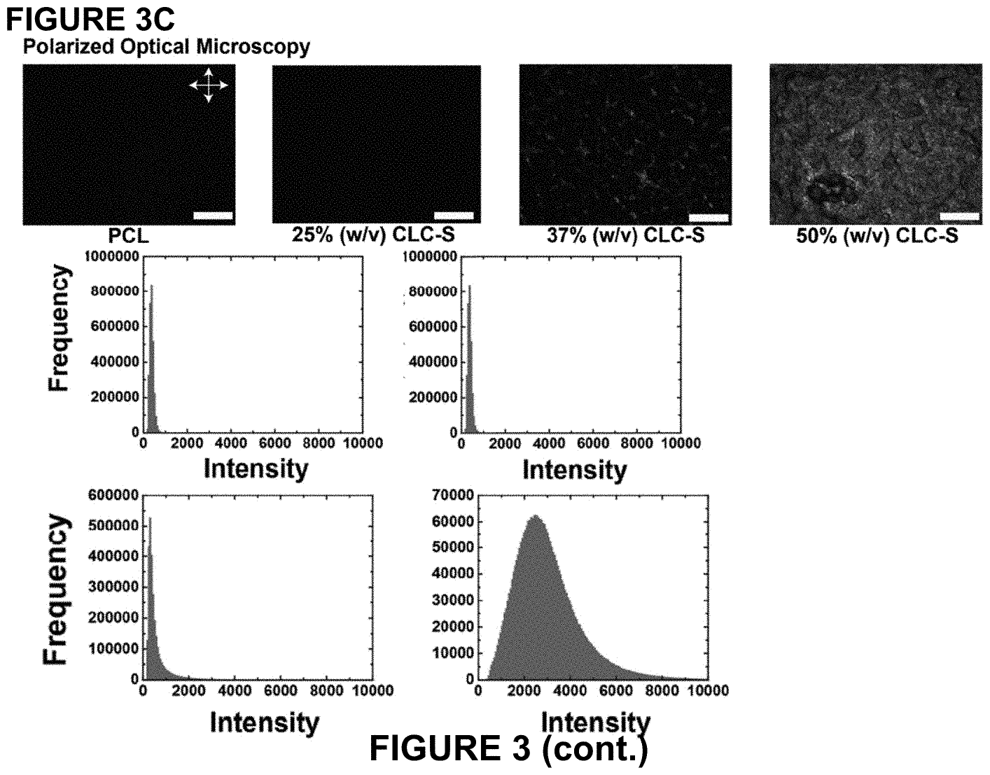

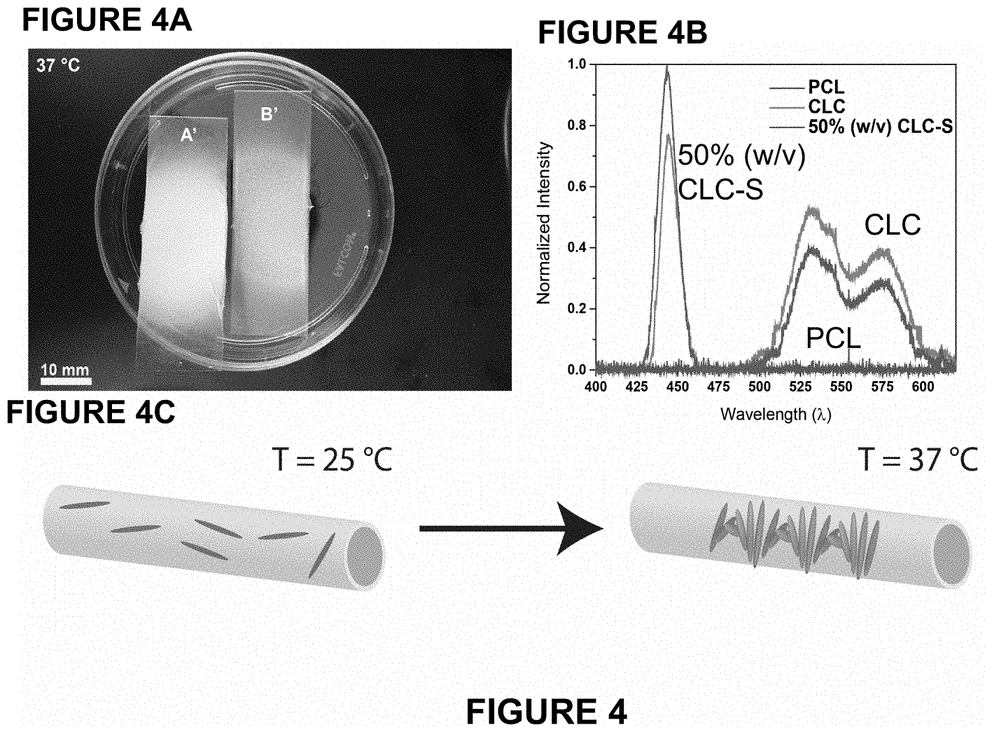

Furthermore, the CLC-S was fabricated via conventional electrospinning and was designed to provide a more fluid-like interface compared to elastic nanofibers typically used in creating synthetic extracellular matrix (ECM) (FIG. 3A). The CLC-S was electrospun with a 15% (w/v) solution of PCL and varying weight per volume fractions of CLC as listed in FIG. 1. Prior to electrospinning, an additive amount of dimethyl sulfoxide (DMSO) was added in order to increase the solution's dielectric constant (c) to enhance fiber formation. The CLC-S with the highest CLC bulk concentration (50% w/v) reflected light off the scaffold when heated to 37 ° C. due to the striations generated by the CLC, as opposed to the scaffolds with lower CLC concentrations, which were opaque (FIG. 3A and FIG. 4). These results indicated that reflection only occurred if the amount of CLC within the polymer matrix exceeded a critical concentration. It was found that the CLC's macroscopic properties were best imparted to the scaffold when the CLC concentration was 50% (w/v).

The UV-Vis spectroscopy results demonstrated that samples electrospun with CLC and PCL (i.e., pristine CLC and 50% (w/v) CLC-S) had similar overall optical reflection profiles when heated to the phase transition temperature, while the nonwoven PCL scaffold controls were opaque. However, a measurable difference was found between pristine CLC (442 nm) and 50% (w/v) CLC-S (443 nm). This shift was likely due to the frustration of the CLC's pitch as a result of its confinement within the PCL matrix (FIG. 4).

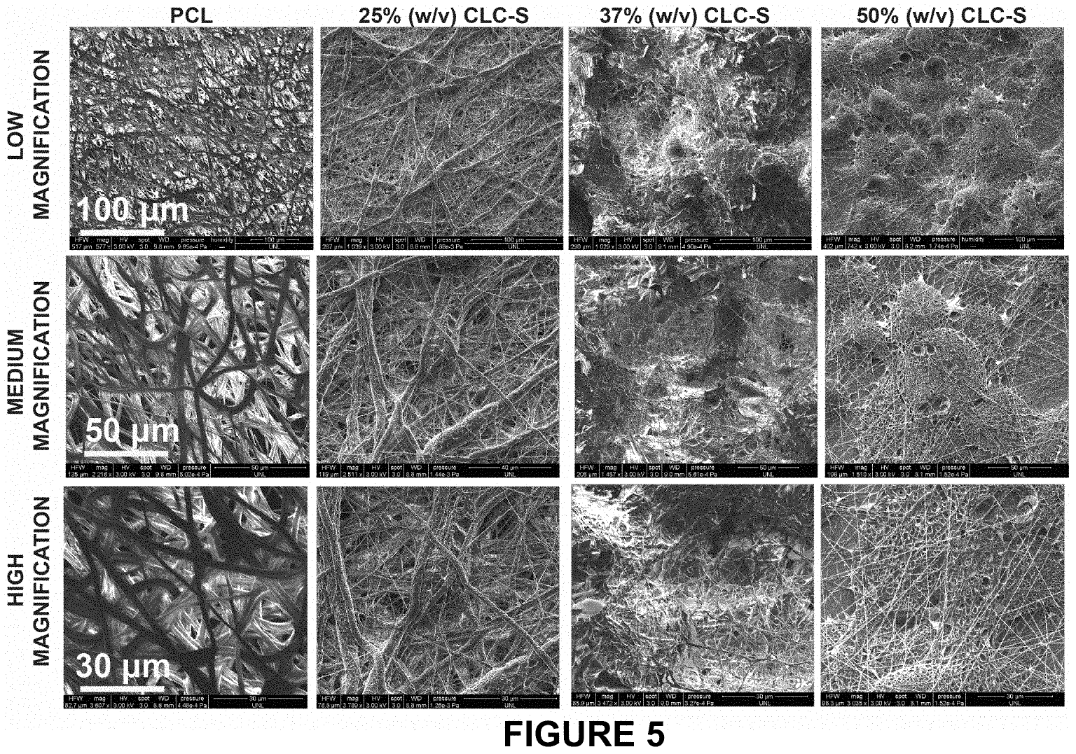

To investigate the features of the CLC-S, CLC-S were imaged using SEM (FIG. 3B and FIG. 5). The PCL-only scaffolds appeared as typical fibrous electrospun scaffolds, and the scaffolds with less than 50% (w/v) CLC demonstrated similar morphology to the PCL-only controls. However, increasing the CLC concentration to 50% (w/v) resulted in regions of aggregation that formed bead-on-string-like morphologies within the PCL polymer network (FIG. 3B and FIG. 5).

To test the encapsulation and functionality of the CLC within PCL network, polarized optical microscopy (POM) was used to measure the light polarizing properties of the CLC-S that were characteristic of CLCs. The CLC-S were first heated to the thermotropic CLC phase transition temperature of 36-40° C. using a custom-built microscope mount heater. While acquiring the 50% (w/v) CLC-S POM micrograph, large regions of birefringent texture polarization across the scaffold were observed(FIG. 3C). These textures were likely due to the confinement of CLC within the PCL polymeric web. These results were also consistent with the previous findings shown in FIG. 3B and FIG. 4.

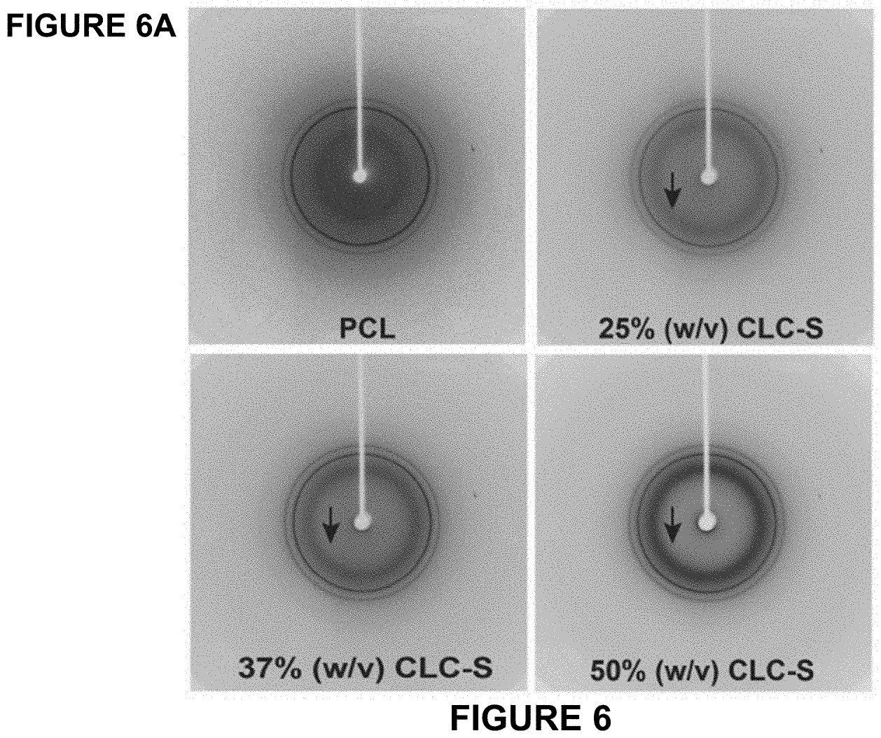

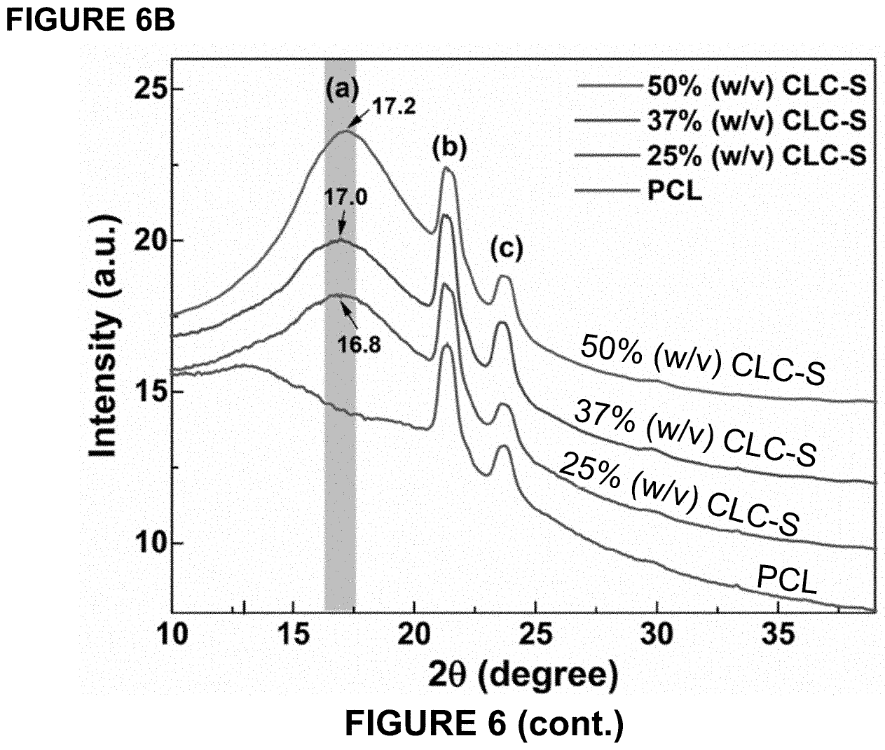

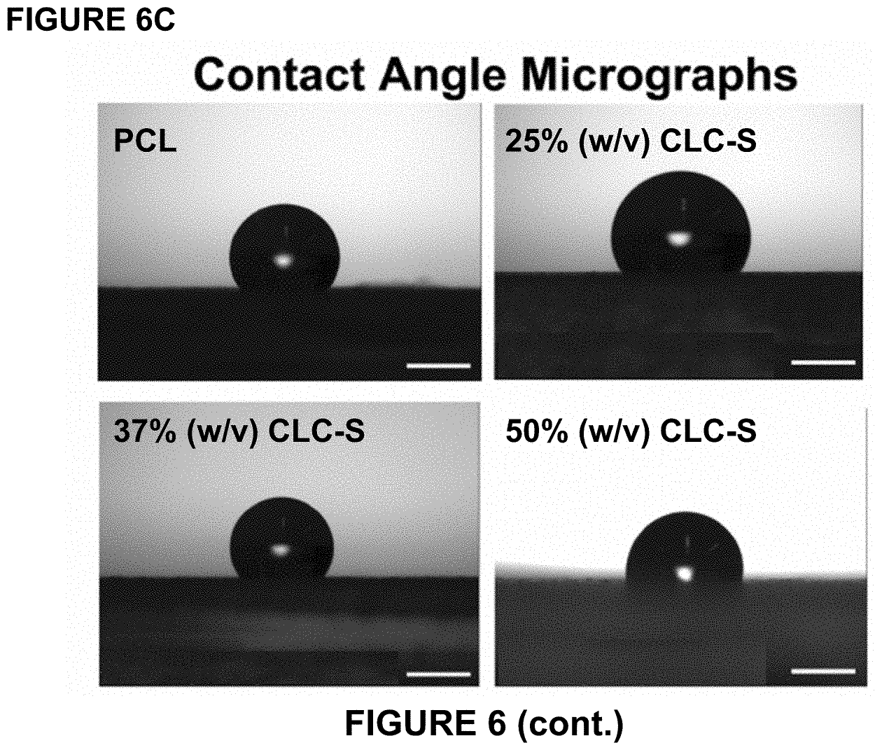

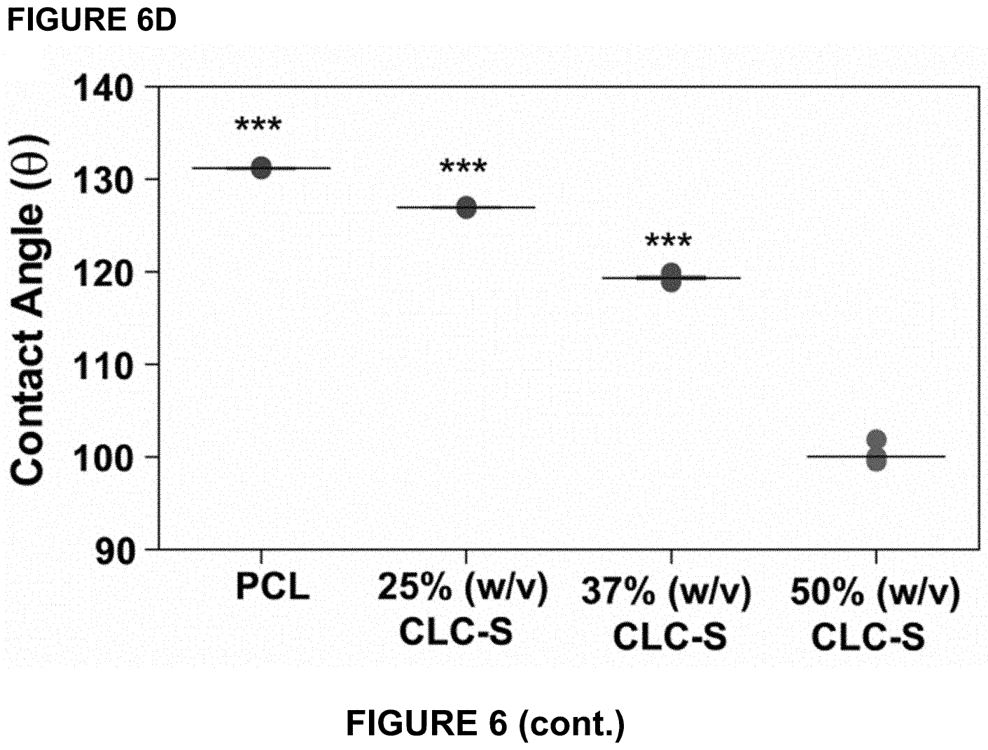

Wide-angle X-ray diffraction further illustrated the incorporation of the CLC within the semi-crystalline PCL mesh (FIG. 6A and FIG. 6B). In the CLC samples, diffuse reflection bands were observed at 2θ=16.8-17.2°, which was similar to what others have reported (Krigbaum W R et al., 1983, Macromolecules, 16:1271-1279). However, these diffuse reflection bands were not present in the pristine PCL scaffolds. As shown in the micrographs in FIG. 6A and subsequent plots in FIG. 6B, gradually increasing the CLC concentration from 25% to 50% w/v shifts the reflection band from 2θ=16.8° to 2θ=17.2°, which corresponded to a decrease in the d-spacing of the CLC from 5.27 Å to 5.15 Å. Moreover, increasing the CLC concentration in the scaffold caused substantial sharpening of the reflection from a full width at half maximum (FWHM) of 6.7° to 4.6°. This change was likely due to the more pronounced bulk properties of the CLC within the polymer network. These results were also consistent with the UV-Vis spectroscopy data, shown in FIG. 4.

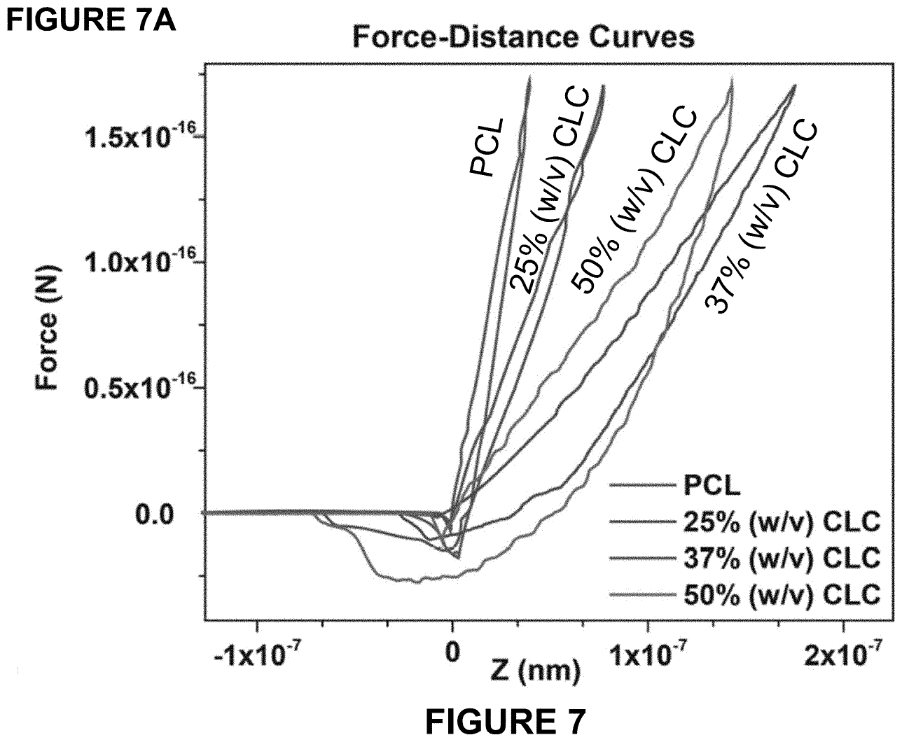

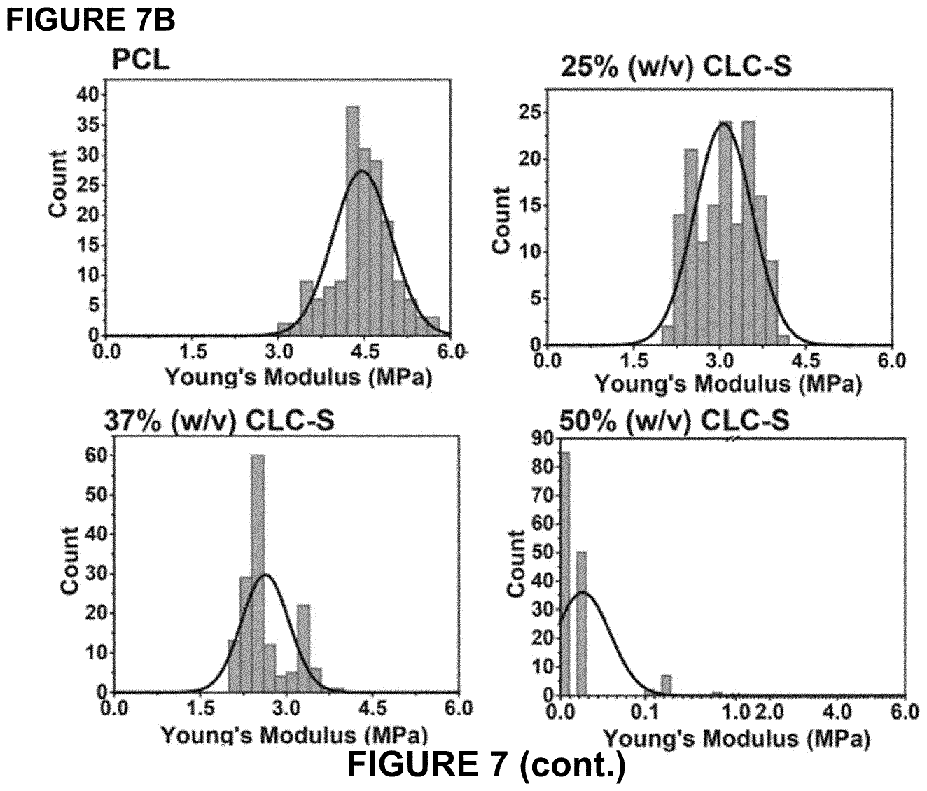

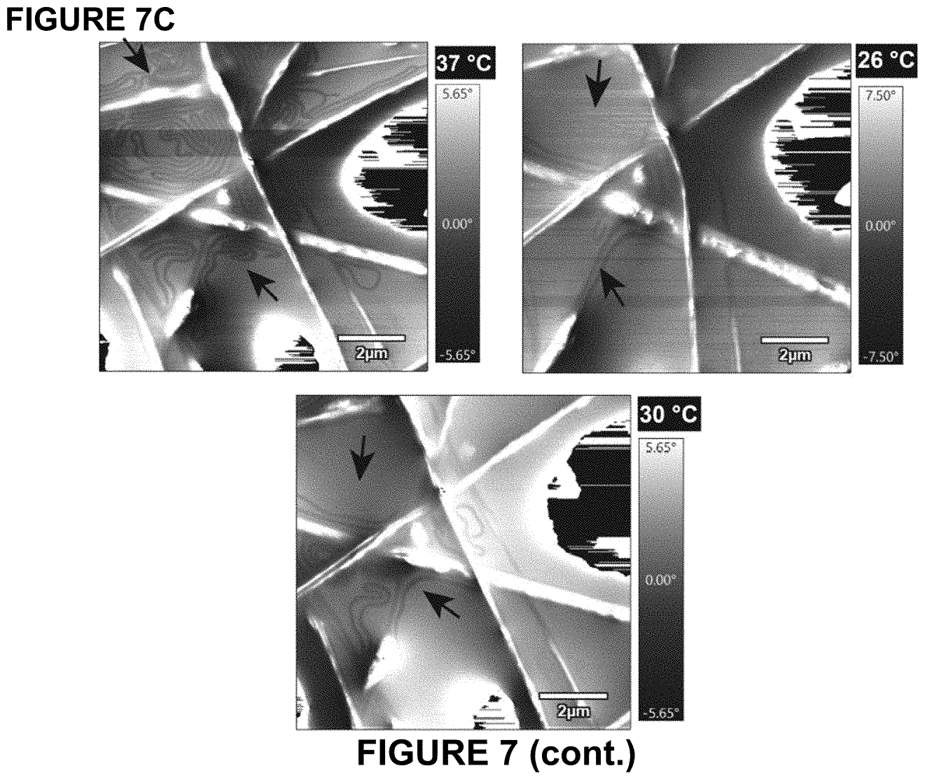

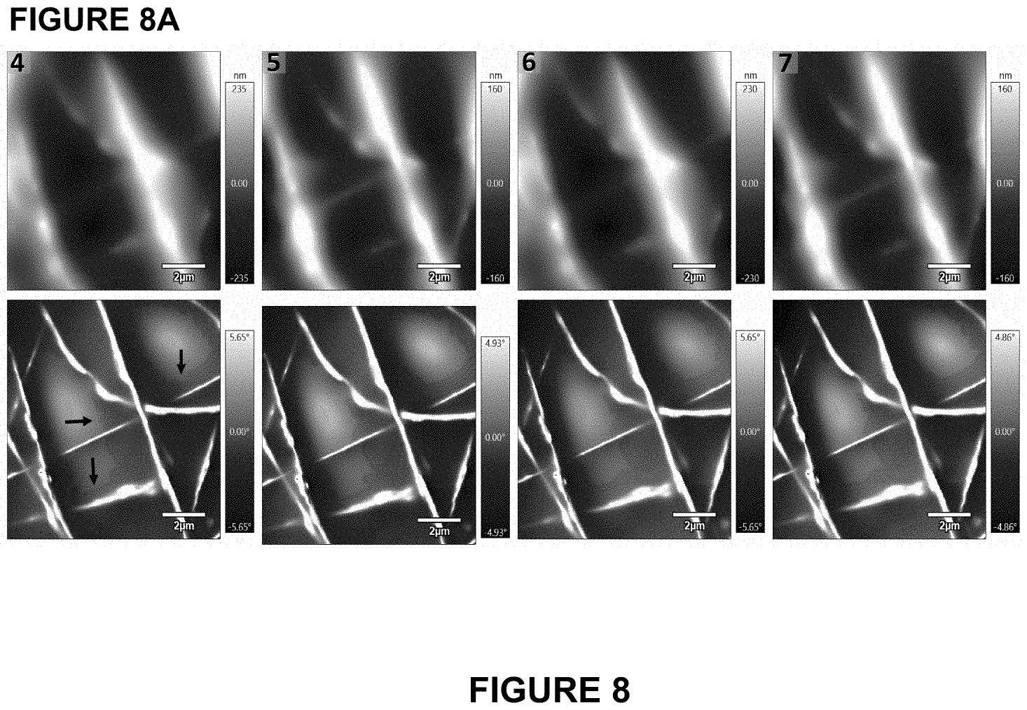

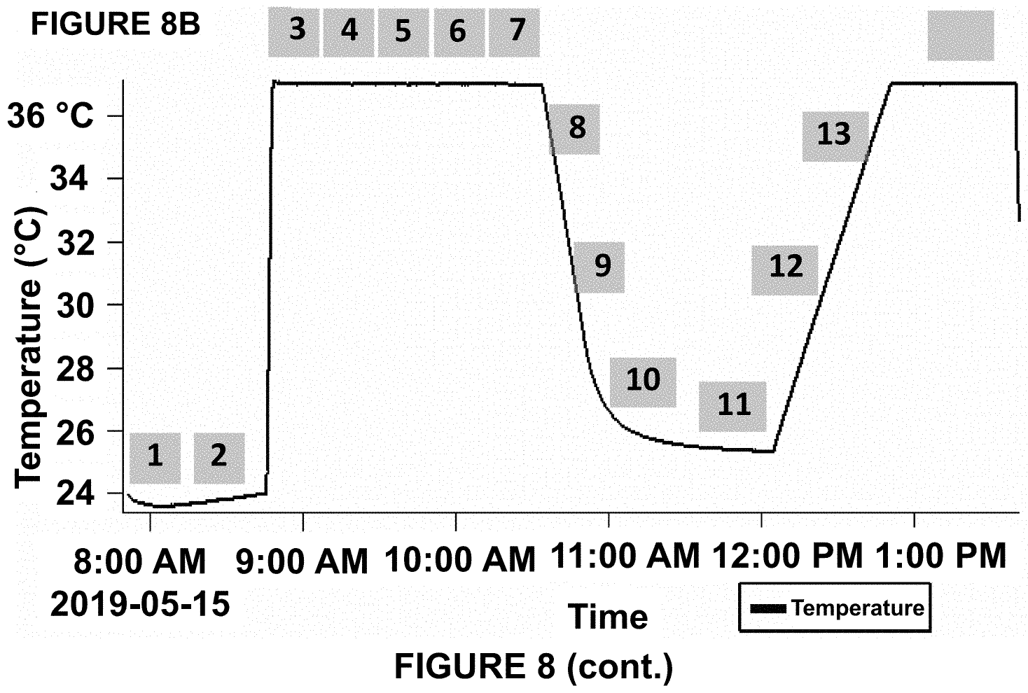

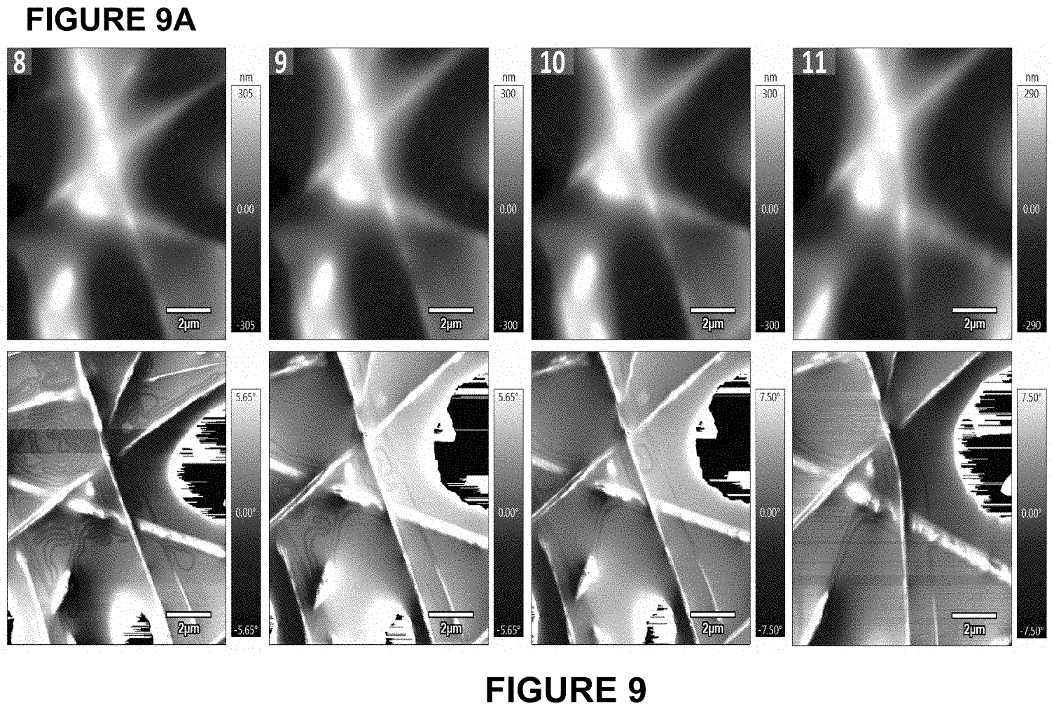

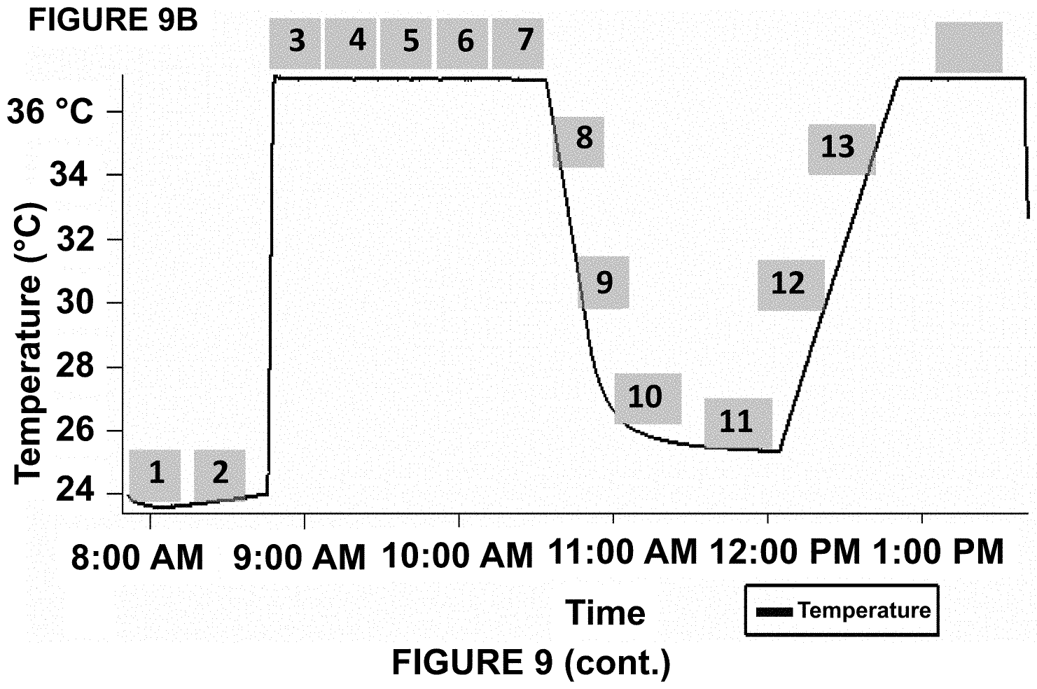

Contact angle measurements were performed to evaluate the overall hydrophilicity of the CLC-S. It was found that increasing the concentration of the CLC rendered the scaffold more hydrophilic (FIG. 6C and FIG. 6D), generating favorable interfaces for cellular attachment and growth (Krigbaum W R et al., 1983, Macromolecules, 16:1271-1279; Ostuni E et al., 2001, Langmuir, 17:6336-6343; Dowling DP et al., 2010, J. Biomater. Appl., 26:327-347). The CLC-S was additional probed with AFM to understand the scaffold's mechanical properties at the bio-interface (FIG. 7A and FIG. 7B). Based on the force-distance curves, elastic deformation profiles were predominantly found for all the tested scaffolds except the 50% (w/v) CLC-S, which demonstrated a viscoelastic deformation profile (FIG. 7A). At 50% (w/v) concentration, the CLC imparted viscoelastic properties within the film and demonstrated thermoresponsive fingerprint-like striations, which were characteristic of cholesteric liquid crystals (FIG. 7C, FIG. 8, and FIG. 9)(Liu D et al., 2014, Angew. Chem. Int. Ed., 53:4542-4546).

Atomic force microscopy was used to map the PCL-only scaffold, 25% (w/v) CLC-S, and 37% (w/v) CLC-S and relatively elastic fiber mechanics with Young's moduli of ˜3 MPa was found. However, when the CLC bulk concentration was increased to 50% (w/v), a softening effect was observed (FIG. 7B). Scanning further, in the 50% (w/v) CLC-S, a non-confined layer of phase-separated CLC was found at the interface of the scaffold that gave rise to fingerprint-like striations upon heating to 37° C. (FIG. 7C, FIG. 8, and FIG. 9). The CLC provided viscoelastic mechanical properties were also seen in collagen (Dyro J F et al., 1975, Mol. Cryst. Liq. Cryst., 29:263-284; Shen Z L et al., 2011, Biophys. J., 100:3008-3015). Interestingly, the 50% (w/v) CLC-S had Young's moduli ranging from ˜3-16 kPa, close to that of muscle tissue (−20 kPa) (FIG. 7B) (Young J L et al., 2016, Exp. Cell Res., 343:3-6). The bulk tensile mechanical properties were further tested with an Instron. There was no compromise in the overall bulk properties upon increasing the CLC concentration, making it suitable for possible in vivo surgical grafting applications that are not be possible using pristine CLCs (which have Young's moduli on the order of pascals) (FIG. 10)(Nasajpour A et al., 2018, Adv. Funct. Mater., 28:1703437; Nasajpour A et al., 2017, Nano Lett., 17:6235-6240.).

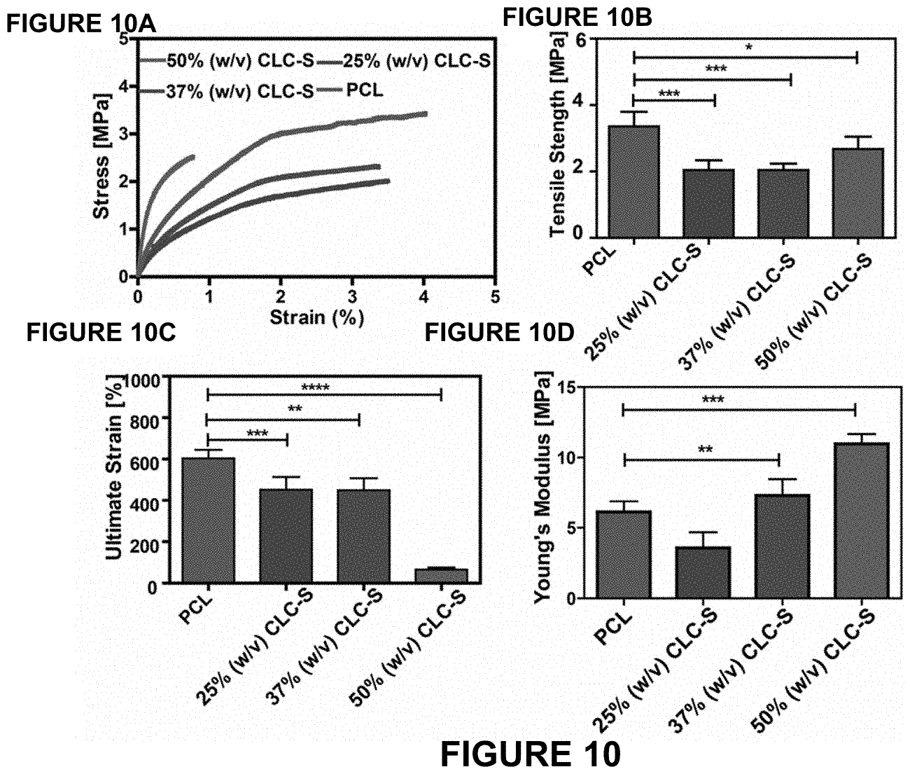

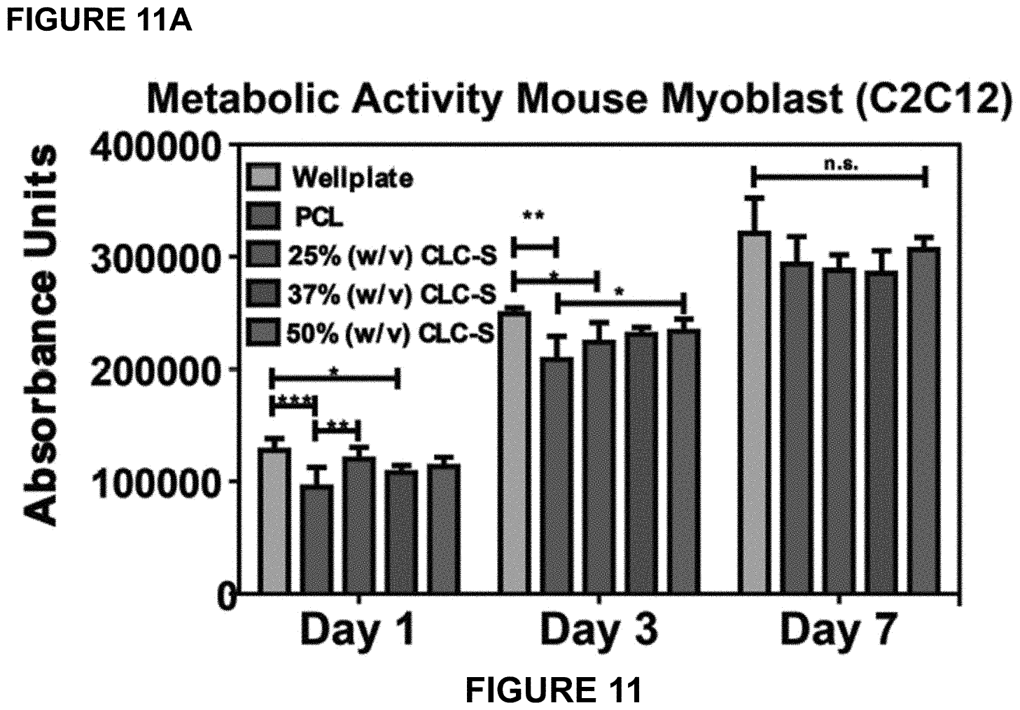

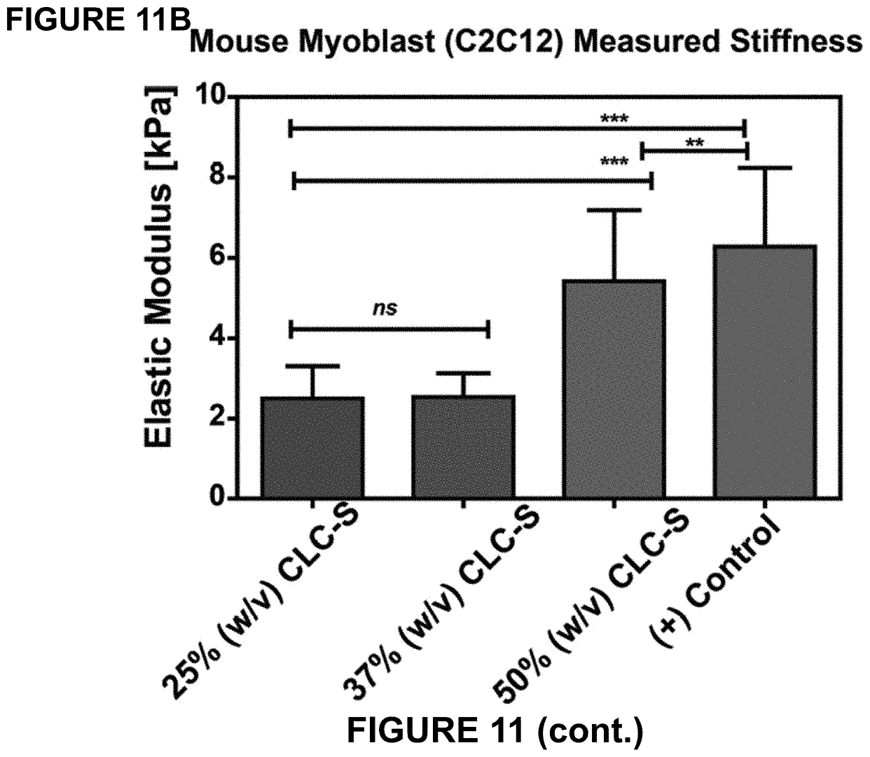

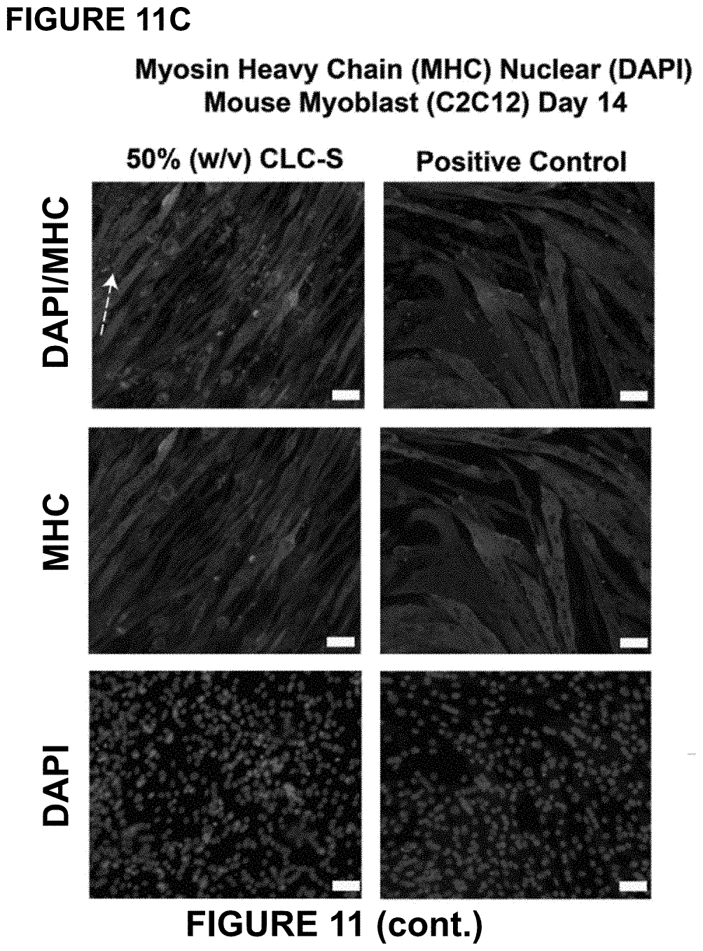

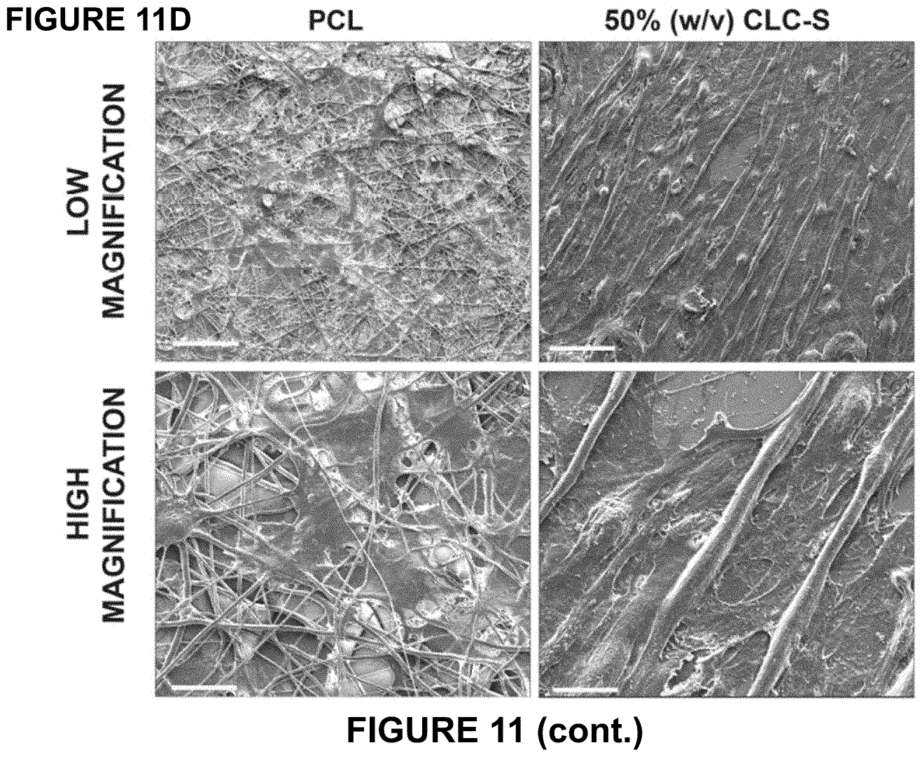

To evaluate the biocompatibility of the engineered CLC-S as tissue engineering scaffolds, mouse C2C12 myoblasts were cultured on them. The CLC-S were analyzed using a cell viability assay kit in fibronectin-free conditions on days 1 and 3 of cell culture (FIG. 11A). The cells remained viable, and the number of cells adhered to the CLC-S increased with CLC concentration (FIG. 12). On the 50% (w/v) CLC-S, the formation of confluent cellular layers was observed, while the PCL-only scaffold poorly supported cellular adhesion, which was likely due to its lack of cellular binding sites (FIG. 12).

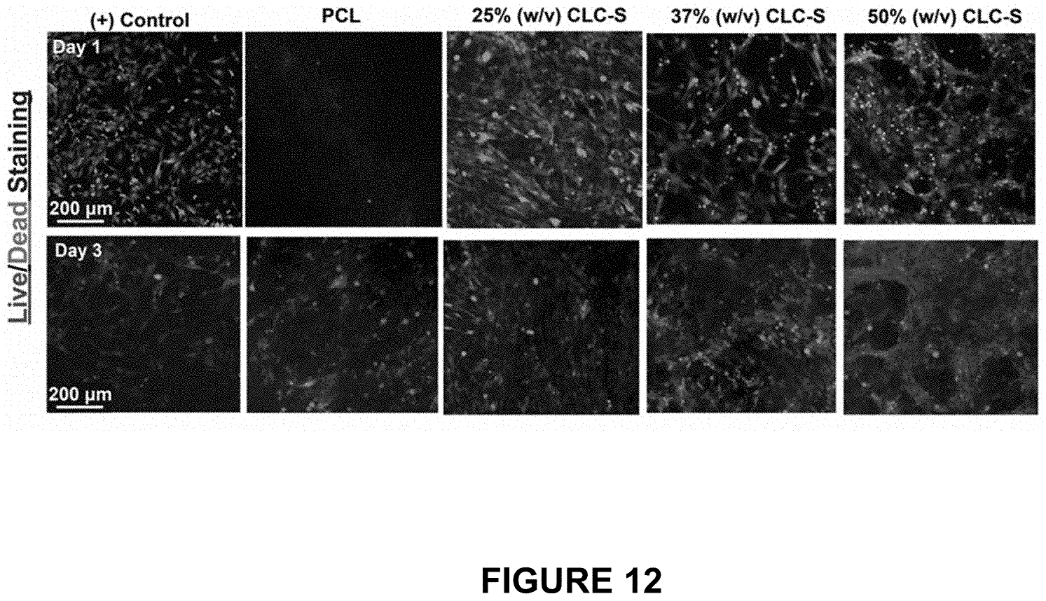

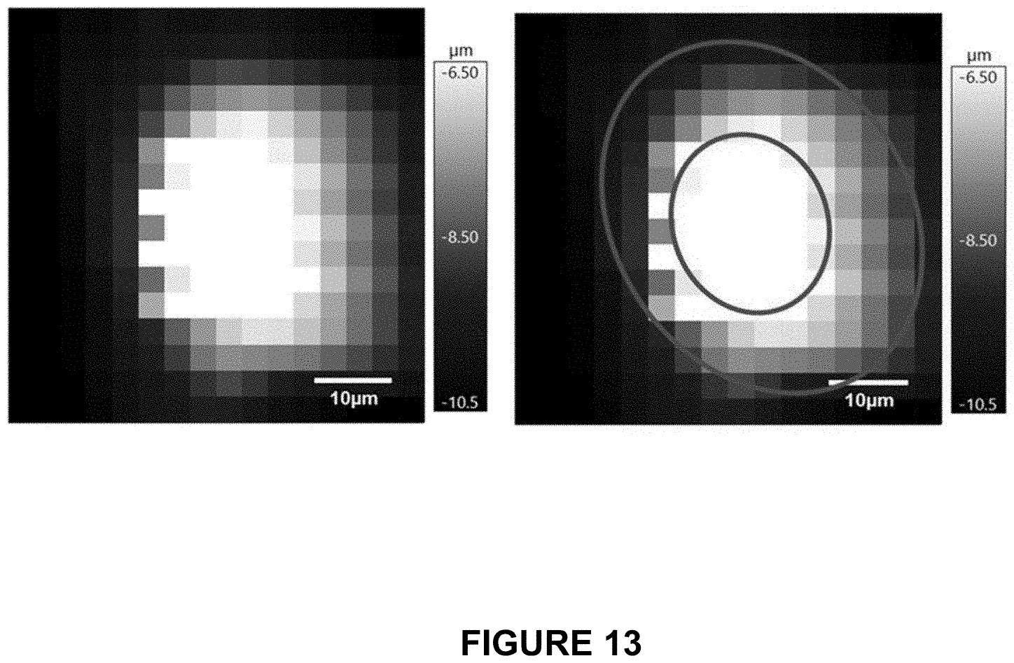

Furthermore, it was observed that increasing the concentration of CLC results in more disclinations as well as favorable viscoelastic properties for cell adhesion through the deposition of soluble cellular factors (FIG. 6C and FIG. 6D). AFM was used to measure the elastic moduli of the mouse myoblast cells adhered to the scaffolds during initial seeding conditions. It was found that the elastic moduli of the cells on the 50% (w/v) CLC-S were significantly higher than those of cells on the 25% (w/v) and the 37% (w/v) CLC-S (FIG. 11B and FIG. 13) indicating cellular binding events. The cellular elasticity measurements indicated that the presence of CLC significantly influenced cellular adhesion and mechano-signal transduction. Interplay between topography, the scaffold's viscoelastic behavior, and wetting of soluble basement membrane proteins excreted from the cultured cells are likely to lead to higher integrin binding activity, remodeling of the actin cytoskeleton, and modulation of cell stiffness (Ghosh K et al., 2007, Biomaterials, 28:671-679; Davidson M D et al., 2019, ACS Biomater. Sci. Eng., 5:3899-3908; Samandari M et al., 2017, J. Biomech., 60:39-47; Babakhanova G et al., 2020, J. Biomed. Mater. Res., 108:1223-1230).

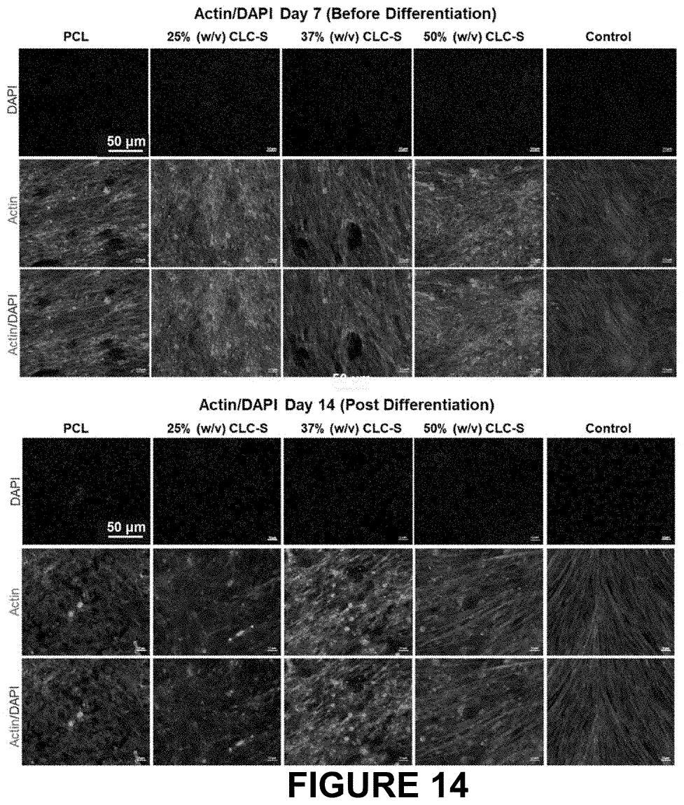

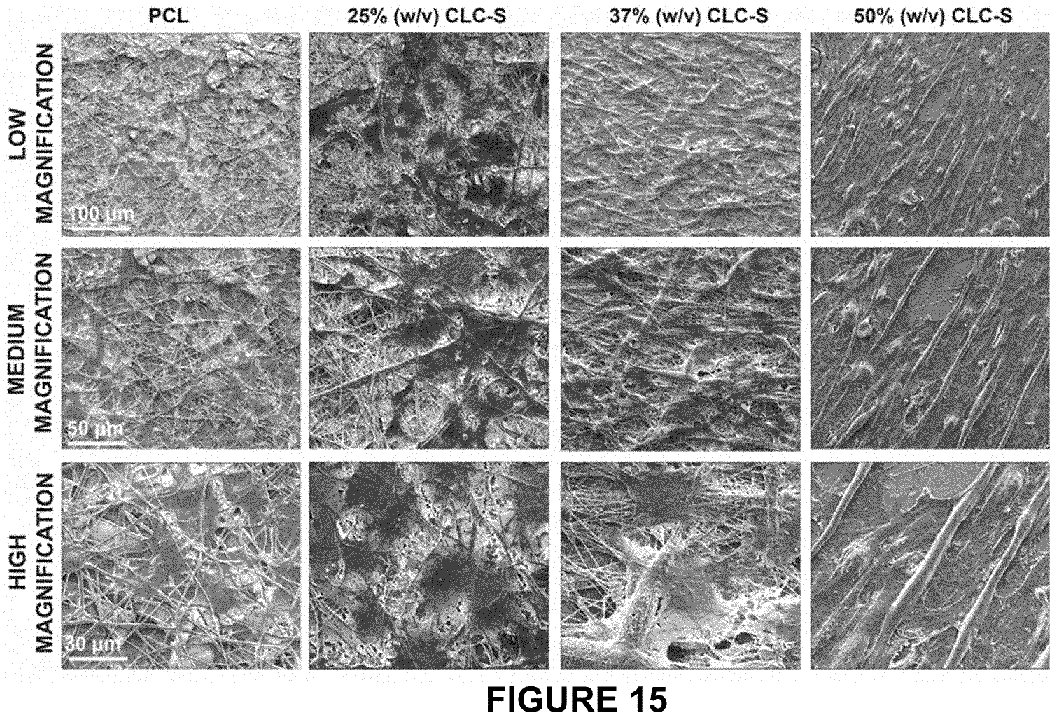

Cellular organization and cytoskeletal arrangement of the cultured cells were then examined by F-actin staining on both day 7 (pre-differentiation) and day 14 post-differentiation. FIG. 14 shows a homogeneous cell distribution over all scaffolds and deeper cellular infiltration with increasing CLC concentration. The F-actin staining data indicated that the addition of CLC did not only generate a suitable environment for cellular attachment but also stimulated cellular infiltration, enabling the formation of 3D-like cellular networks. Myoblast differentiation to myotubes were confirmed by immunostaining against myosin heavy chains. Immunohistochemical analyses demonstrated myoblasts seeded on the 50% CLC-S on day 14 exhibited formation of multinucleated elongated myotubes with positive myosin heavy chain expression, comparable to positive controls (FIG. 11C and FIG. 15).

The myotubes' formation was further confirmed on 50% (w/v) CLC-S with SEM images of the scaffolds (FIG. 11D). At 50% (w/v) CLC-S, elongated myotubes were formed, indicative of cellular organization, despite the random alignment of the scaffold. The thicknesses of these myotubes had a direct relationship with the CLC and its concentration (FIG. 11D and FIG. 15). Overall, the combination of the CLC viscoelastic mechanical properties, biocompatibility of mesogens, and the topologic disclinations of the CLC-S facilitated the deposition of soluble cellular attachment factors, which facilitated cellular adhesion, proliferation, and differentiation.

In summary, a CLC-S with viscoelastic mechanical properties was developed, which provided an environment for the growth of mouse myoblast cell lines. The advantages of the herein-described scaffold were its straightforwardness, biocompatibility, and robust mechanical properties. The scaffolds had both the strength to withstand surgical procedures and mechanical properties that mimicked those of the native muscle ECM. Additional studies focus on elucidating the interaction mechanisms between soluble protein factors and the CLC-S to include quartz crystal microbalance measurements to monitor the time course of soluble protein attachment accompanied with genetic analyses of activated mechanosignal transduction through integrin-binding mechanisms to understand cellular attachment. In parallel, the effects of the helicity of the cholesteric phase on protein attachment are also leveraged to serve as a treadmill for the cultured cells. Additional studies also involve fabrication of 3D scaffolds to culture multiple human cell types to work in concert as functional muscle tissue.

In conclusion, liquid-crystal-based biomaterials provided platforms for the development of dynamic and responsive interfaces for tissue engineering. The herein-described CLCs were particularly well suited for these applications, due to their roles in cellular homeostasis and their intrinsic ability to organize into supramolecular helical structures that reflected light in reversible manner at a critical phase transition temperature. More specifically, the present studies described the development a nonwoven CLC electrospun scaffold by dispersing three cholesteryl ester-based mesogens within fibrous networks. The fibrous materials can be made from any polymer (e.g., any edible or food grade polymer). The ratio of the CLC mesogens was judiciously tuned to achieve a phase transition at the cell culture incubator temperature of 37° C. In these scaffolds, the PCL provided structural integrity while the homogenously dispersed CLCs adopted a helical structure with its axis parallel to the surface, causing the mesogens to alternate between homeotropic and planar alignments. This alternating orientation gave rise to fingerprint-like striations that established a dynamic interface, which mimicked the hierarchical and dynamic structures found within the native ECM in skeletal muscle tissue. Using AFM as well as immunochemistry, it was found that these fingerprint-like striations led to greater myoblast adhesion strength, shortened differentiation times, and overall enhanced myofibril formation in vitro compared to the tested control counterparts.

The materials and methods used in this example are described below.

### Materials

Chloroform, dimethylformamide (DMF), cholesteryl oleyl carbonate, cholesteryl pelargonate, and cholesteryl benzoate were purchased from Sigma-Aldrich (St. Louis, Mo.). Cell culture media were obtained from Gibco (Gaithersburg, Md.) and all cell culture reagents were purchased from Fisher Scientific (Agawam, Mass.).

### Electrospinning of the CLC-S

In brief, polycaprolactone pellets (Mn=80,000) and the three cholesterol derivatives used in the study are added to a scintillation vial: overall composition was 15% (w/v) PCL polymer and dispersed 25%, 37%, and 50% (w/v) CLC phase concentration. (Table 1) The overall solvent concentration is a (3:1) azeotropic solution consisting of three parts chloroform to one-part DMF. The reagents are solubilized in chloroform and then DMF is added to increase the solution's permittivity. The CLC-doped polymer solution is then transferred to a 3 mL plastic BD syringe with a 20-G blunt needle tip. The solution is loaded on a syringe pump set at a rate of 3 mL/h with an overall applied electrical voltage at 15 kV at a fixed total distance of 18 cm at 25% atmospheric humidity. Samples were collected for 15-20 min per condition.

### Scanning electron microscopy analysis of the CLC-S

The fiber morphology was investigated using a scanning electron microscope (SEM; Zeiss 40P Ultra Plus). The samples were electrospun directly on a gold silicon wafer and imaged under variable pressure with low voltage conditions.

### Polarized optical imaging analysis of the CLC-S

Polarizability of the CLC-S was tested with a polarized optical microscopic (POM; Zeiss Imperio, Jena, Germany). The samples were directly electrospun on a clean glass slides to reduce unnecessary background. Micrographs were taken with a 50 ms exposure time for all samples with measurements collected at 37° C. with 25% atmospheric humidity.

### Mechanical characterization of the CLC-S

The mechanical properties of the prepared scaffolds were measured using a uniaxial tensile strength test with a universal mechanical testing machine (Instron 5542, Norwood, Mass.). Four samples were cut in a ribbon rectangular-shaped (20 mm×5 mm). The constant crosshead speed was set at 15 mm/min and the related force was measured using a lON cell (n=4).

### Atomic Force Microscopy Method for Mechanical Evaluation of the CLC-S

The CLC nanofibers were imaged with an MFP 3D BIO (Asylum Research/Oxford Instruments, Goleta, Calif.) atomic force microscope. To map the topographical features with phase contrast images to probe the electrospun fibers liquid crystal effect on the fibrous structure, silicon AC240 TS (Olympus) scanning probes was used at 0.3 Hz with a nominal spring constant of 2.7 N/m and the radius <10 nm. Prior to imaging, the probe's drive frequency was calibrated via the instrument's Auto Tune function. Imaging of the electrospun fibers in 20 randomly selected areas was performed in Semi-Contact mode, with an enabled phase contrast data acquisition. Topographic data analysis was performed with IgorPro (ver. 6.17), described previously. S1 The microscope is housed on an anti-vibration table enclosed in an anti-acoustic chamber. The scanning environment was set at a temperature (ca. 37° C.) and an air humidity (ca. 28%) was controlled by an external air-conditioning system.

Mechanical evaluation of electrospun scaffolds at the nanoscale was conducted with the same atomic force microscope equipped with silicon-nitride RC 800 PSA (Olympus) scanning probes, with a spring constant of 0.8 N/m and tip radii of 30 nm. The spring constant of the cantilevers were calibrated during the experiments using a custom-made silica standard. Images were collected in Force Spectroscopy (FS) mode to produce registration of maps of mechanical (stiffness) properties. The force-distance curves were generated from maps of 32×32 points. To avoid artifacts caused by the sample's curvature, only the highest regions (on the top of the 25 fibers) were chosen for further analysis.

The mechanical evaluation of the C2C12 mouse myoblast seeded on electrospun mats was performed using the AFM mentioned prior, operating in FS mode. To avoid damage to the cells, a tipless scanning probe NP-010-D (Bruker) with a spring constant of 0.03 N/m was chosen for the experiment. All measurements were collected in an aqueous cell culture media, 6 h after cells seeding, all the measurements were taken within 1 h of removing the samples from the incubator. Each C2C12 cell was localized with the AFM's integrated optical system, the cantilever was positioned central centrally above the cellular nucleus during measurements. IgorPro (ver. 6.17) software was used to analyze the mechanical data. Selected raw data were transformed into force versus indentation curves. Johnson-Kendall-Roberts (JKR) theory was chosen for fitting experimental data used to calculate stiffness of the electrospun mats, as described previously. S1-S3 (n=4)

### Two-Dimensional X-Ray Diffraction Analysis of CLC-S

The CLC fibrous scaffolds were loaded into the sample holder in ribbons and were mounted vertically to the monochromatic Cu X-ray beam of an Oxford X Calibur PX Ultra diffractometer equipped with a low noise CCD Onyx area detector. The detector was calibrated using a calcium carbonate reference with a reflection at [104] calculated to 3.035 Å. The diffraction spectra generated for each condition were collected under identical experimental conditions.

### Contact Angle Measurements of CLC-S

Contact angle measurements were performed on scaffold electrospun directly onto glass slides using a goniometer (KSV CAM 101) at room temperature. The droplet profiles were captured with 5 μL water droplets placed on different regions of the scaffolds. The droplet profiles were captured and were used to calculate the contact angle values (n=6).

### Cell Culture and Cell Viability Assessment

Mouse myoblast cell line (C2C12, Sigma-Aldrich) was cultured in Dulbecco's modified eagle medium (DMEM) (Fisher Scientific, Agawam, MA) supplemented with 10% (v/v) FBS (Gibco) and 1% (v/v) penicillin-streptomycin (Thermo-Fisher, Bedford, Mass.). Cells were passaged roughly every 3 days at 80% confluence. Fibers that were spun directly on coverslips (18 mm×18 mm), were sterilized with UV light for 5 min and placed in well plates. Samples were seeded with 15,000 cells.

The viability of the cultured cells was assessed on day 1 and 3 using a Live/Dead™ Viability/Cytotoxicity Kit (Invitrogen) according to the manufacturer's instructions. In brief, samples were incubated with a solution of 0.5 μL/mL calcein-AM and 2 μl/mL ethidium homodimer in PBS for 10-15 min at 37° C. and immediately imaged with a Zeiss Observer fluorescence microscope. In the images, live cells appear as green while dead cells can be seen on the red channel. Cellular metabolic activity was investigated via the PrestoBlue Cell Viability Assay (Invitrogen) as per the manufacturer's instructions. The changes in cellular metabolic activity were correlated to the cell count and the results were used to estimate cellular proliferation. The analysis was done on cells that were directly cultured on the samples and also on cells that were cultured in well plates and then seeded onto the samples (n=6). PrestoBlue assays were conducted on days 1, 3, and 7 of culture. In brief, at each time point, cells were incubated with culture medium containing 10% (v/v) of the assay reagent for 1 h at 37° C. The fluorescence intensity of the solution was measured using a Citation 5 spectrophotometer (BioTek, Vinooski, Vt.) at 540 nm excitation and 600 nm emission.

### Assessment of Cellular Differentiation

Directed differentiation of cultured myoblasts was initiated by changing the culture medium to a differentiation medium, which consisted of DMEM supplemented by 2% (v/v) horse serum (Gibco) and 1% (v/v) streptomycin-penicillin (Thermo Fisher), on day 7. The samples were kept under these conditions for an additional 7 days with media exchanged every 3 days. The samples were fixed on day 7 and day 14 with 4% (w/v) paraformaldehyde and permeabilized with a 0.1% (v/v) Triton X-100. To visualize cellular morphology and cytoskeleton, F-actin was stained with Alexa Fluor™ 488 Phalloidin (Invitrogen) according to the manufacturer's instructions for 30 min. Cellular differentiation was confirmed by staining against myosin heavy chain (MHC) (ab51263) used as a primary antibody. A labeled Goat Anti-Mouse IgG H&L (Alexa Fluor® 488) (ab150117) was used as a secondary antibody. Samples were washed 3 times with PBS after permeabilization with 3% Triton and were incubated with a blocking solution consisting of 1% (w/v) bovine serum albumin (BSA), 0.1% (v/v) Tween and 22.52 mg/mL glycine. Samples then were incubated with the primary antibody overnight and then with secondary antibody for 1 h. Samples were imaged using the same Zeiss microscope.

### Statistical Analyses

The experiments and measurements were carried out at least in triplicate, and the data are reported as mean values±standard deviation. A one-way ANOVA test was used for compared the groups, and the values were considered statistically significant different when p values resulted lower than 0.05: *p<0.05, **p<0.01, ***p<0.001.

### Example 2: CLC Cell-Laden Microcarriers for Bioreactor Culture

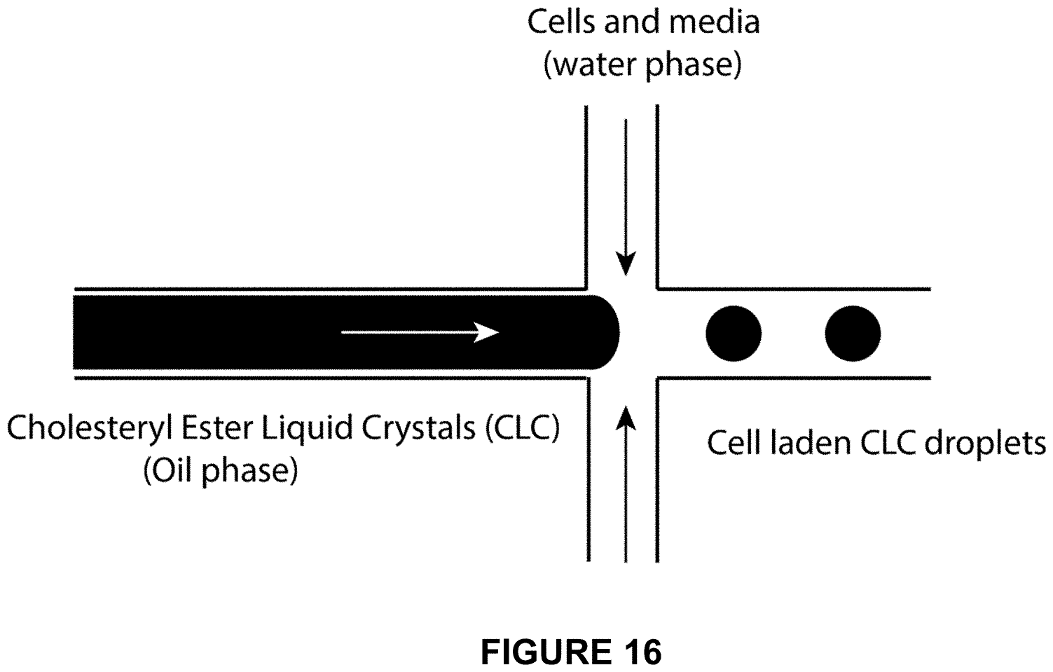

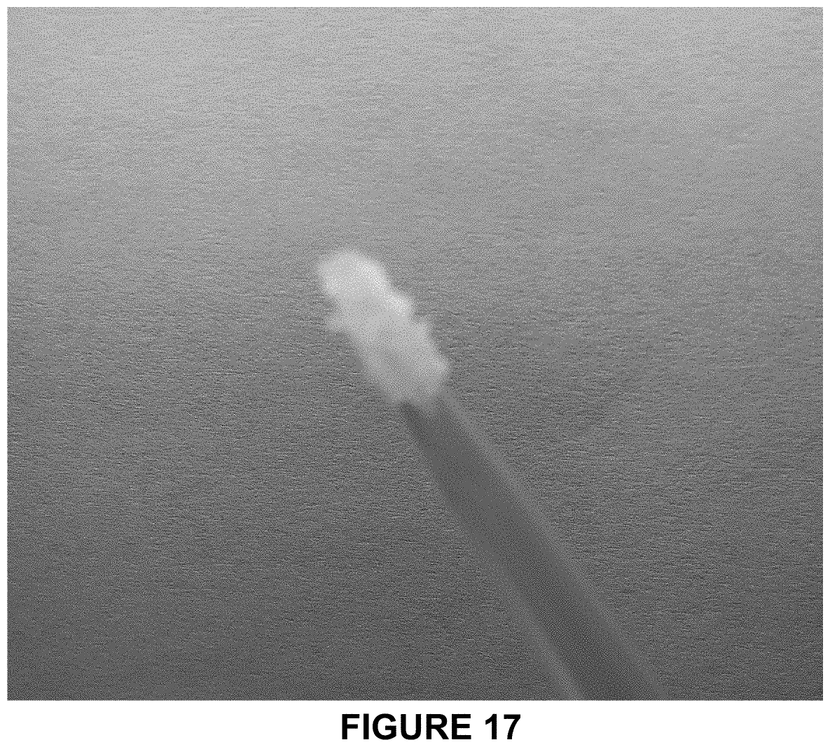

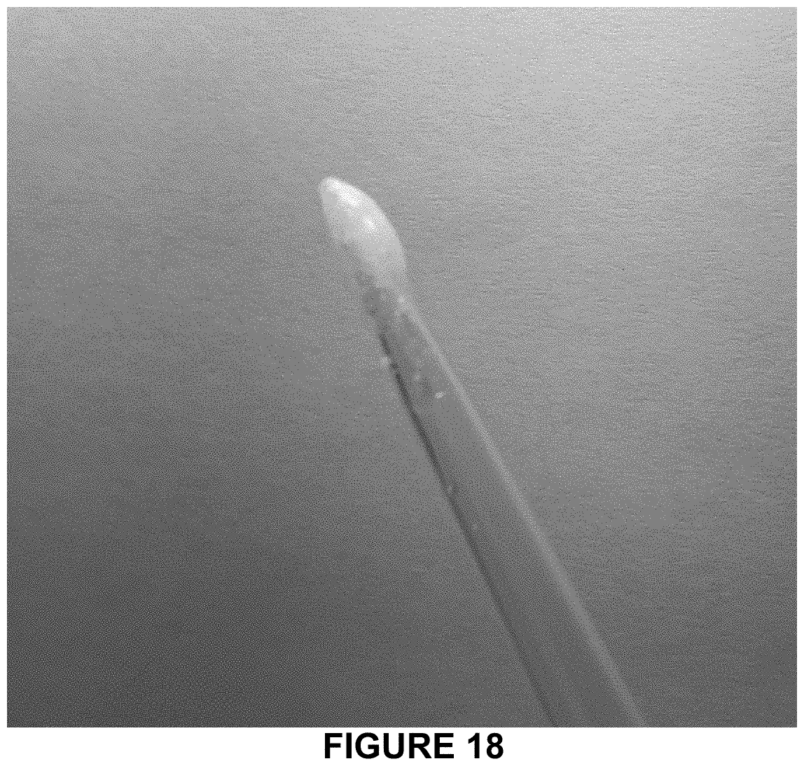

To generate CLCs cell-laden microcarriers for bioreactor culture, the CLC flow in the melt as the oil phase while the water phase comprises cells of interest for “clean meat” production (e.g., avian, bovine, fish, porcine, etc.) (FIG. 16). This method generates freestanding droplets of CLC with the cells of interest. The CLCs allow for cellular attachment and inhibit program cell death, anoikis. More importantly, commonly used polystyrene beads (Corning Microcarriers) implemented in cellular bioreactors are coated with the CLC material (FIG. 17 through FIG. 19). To enhance cell-binding events, as polystyrene beads contain no cell-binding instructions for cellular attachment, the manufacturer provides collagen or hydrophilic microcarriers and other propriety technology unsuitable for clean meat production. Furthermore, this method is not limited to standard microdroplet generators. For example, electrospraying methodology using a low molecular weight aliphatic polymer less than MW=40,000 (PLGA, PCL, PS, etc.) combined with CLC produce microcarriers of interest. Usage of low molecular weight polymers inhibits fiber production and generates microcarriers. Furthermore, 3D printing methodology using a coaxial needle generates CLC droplets of interest with and without cells.

### Example 3: CLCs for the Growth of Muscle Cells

Three cholesterol mesogens (cholesteryl oleyl carbonate, cholesteryl pelargonate, and cholesteryl benzoate) were melted using a heat gun in a vial. When the mesogens were fully melted at 70° C., the melt was colorless. Once the mesogen equilibrated to room temperature, it formed the CLC material that was coated on the standard well plate.

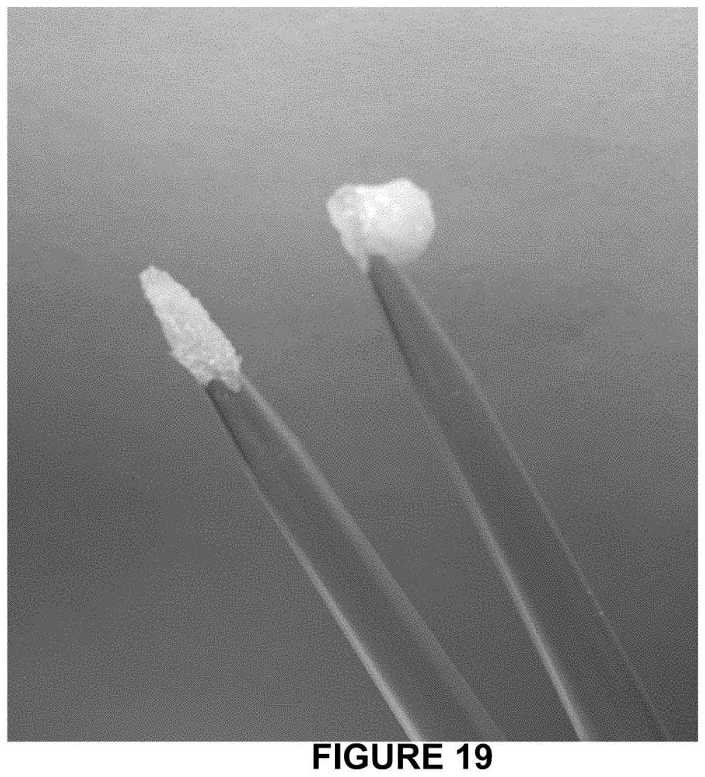

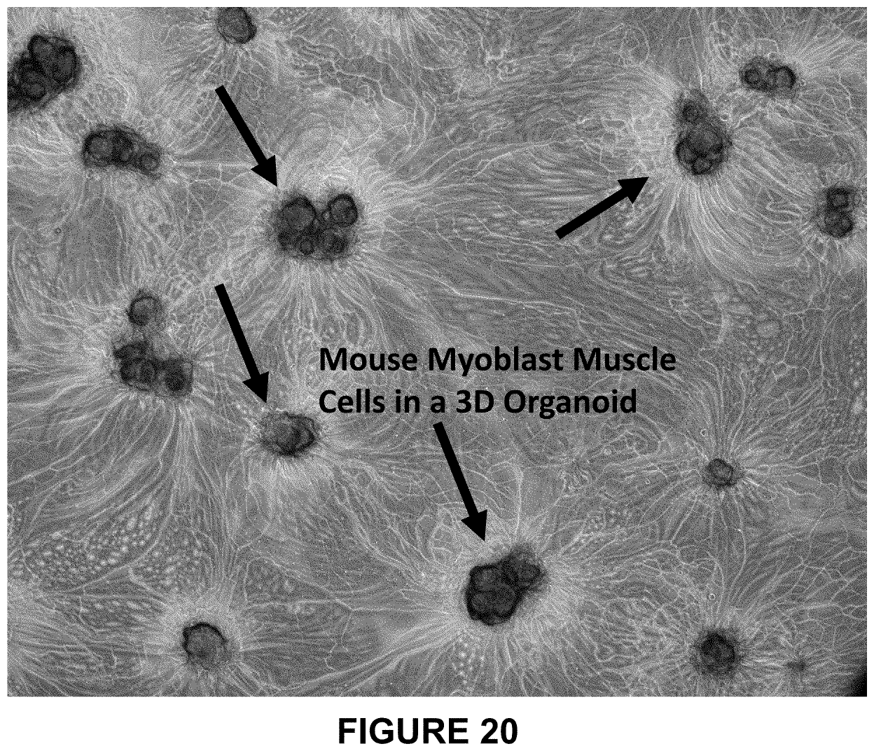

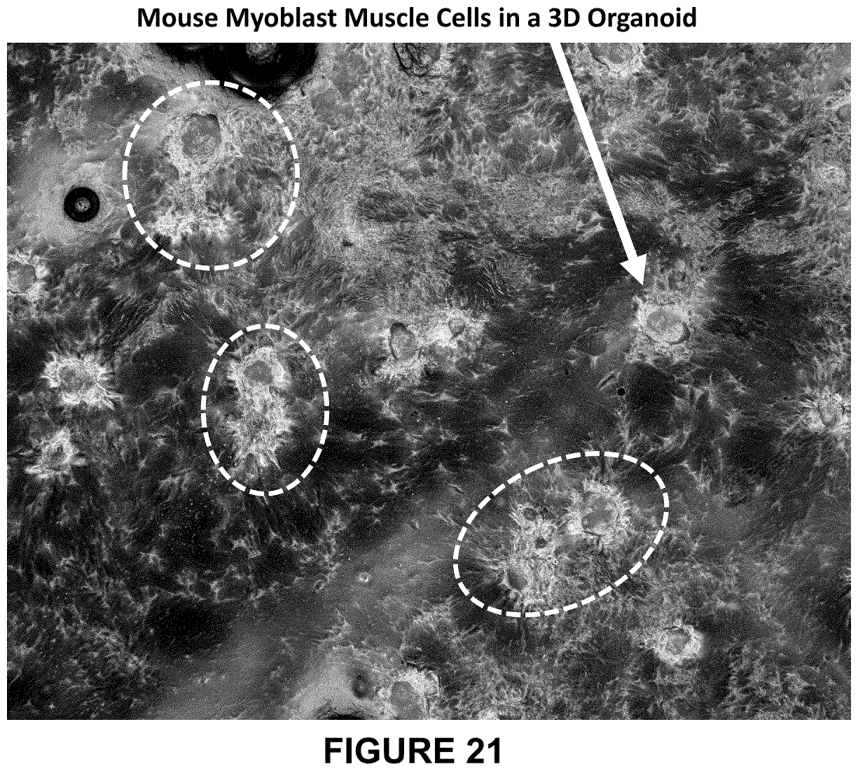

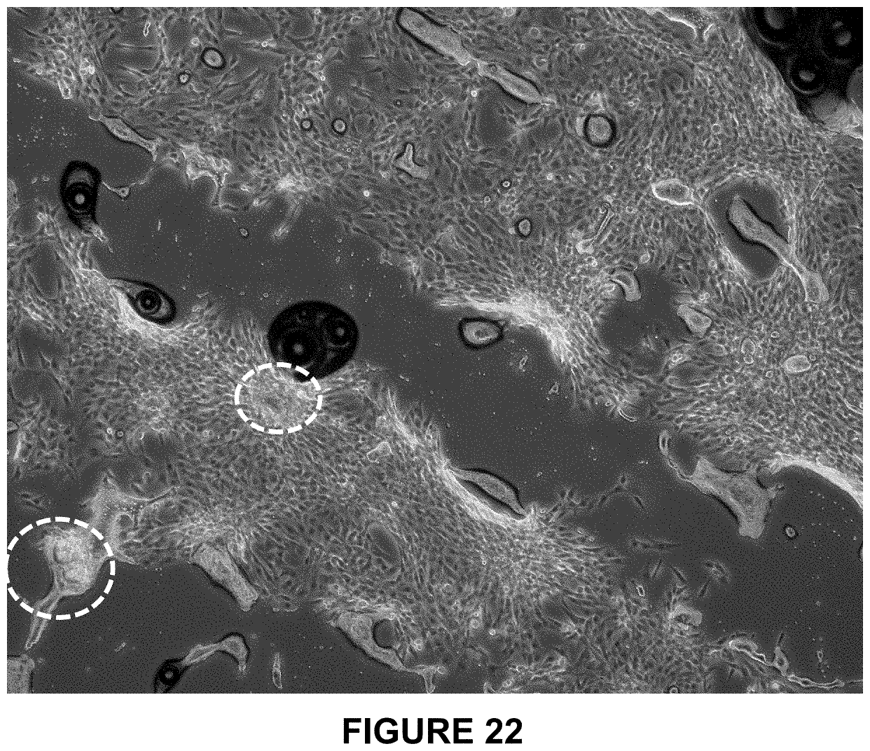

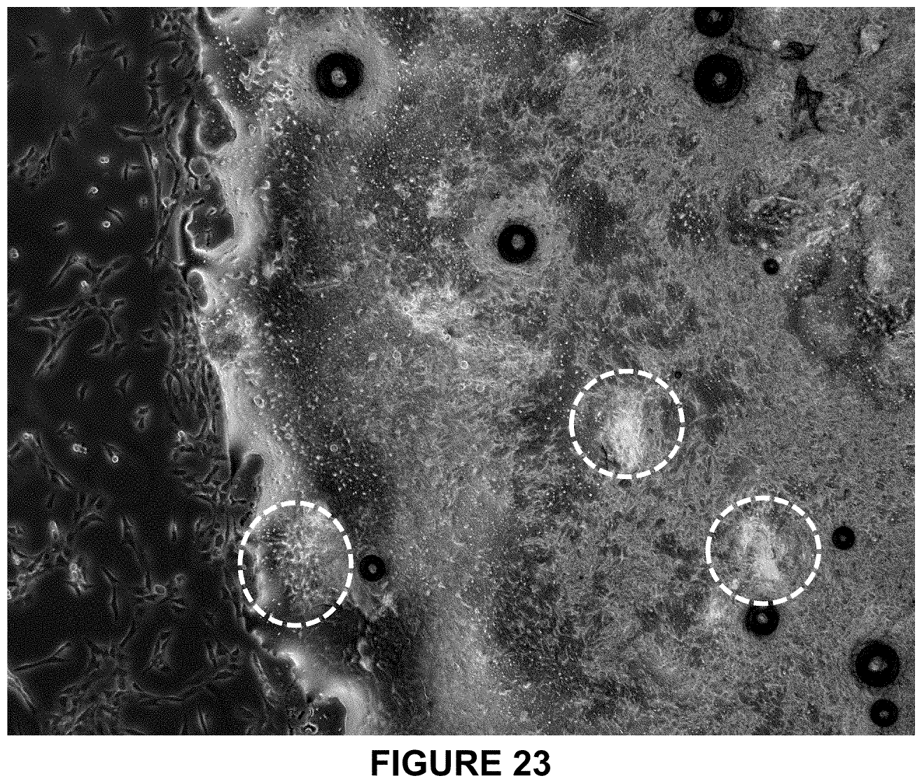

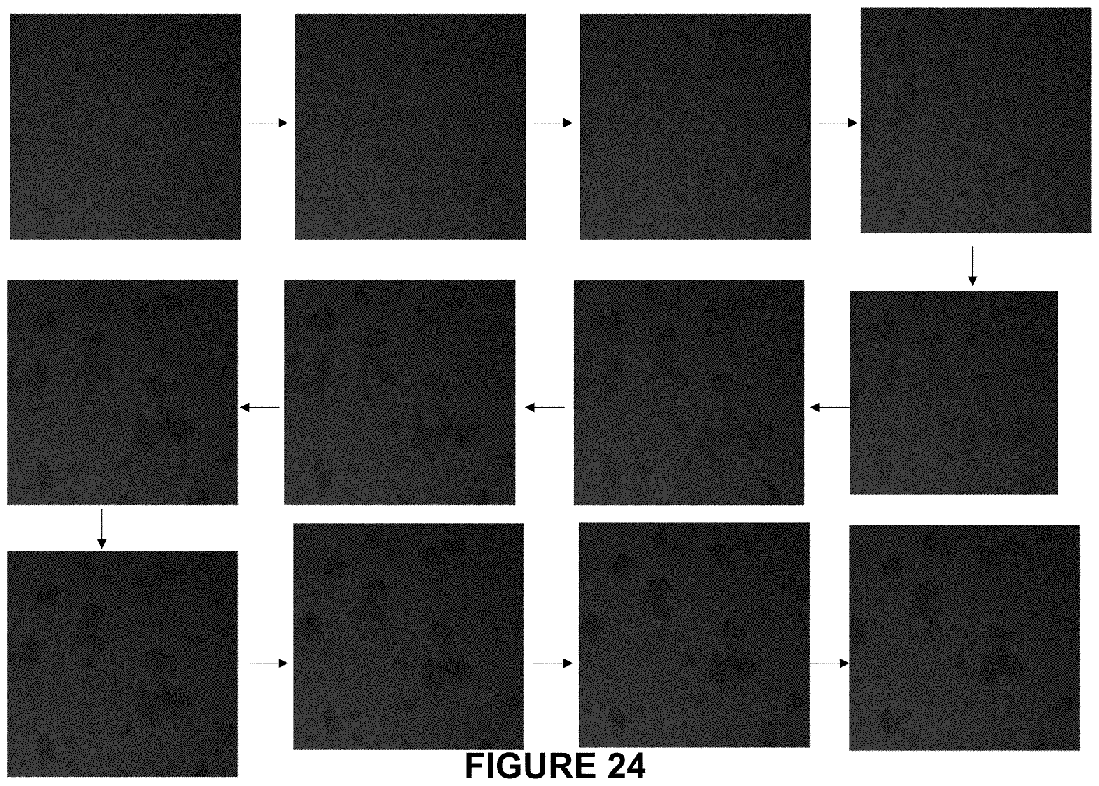

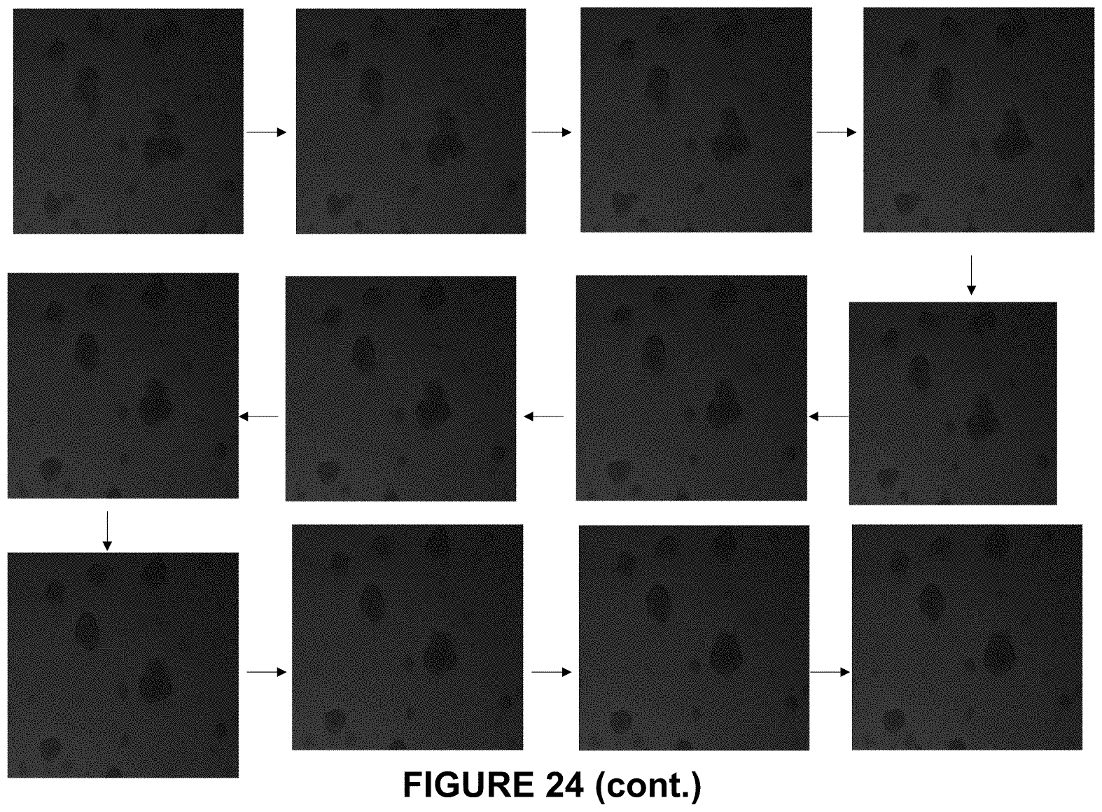

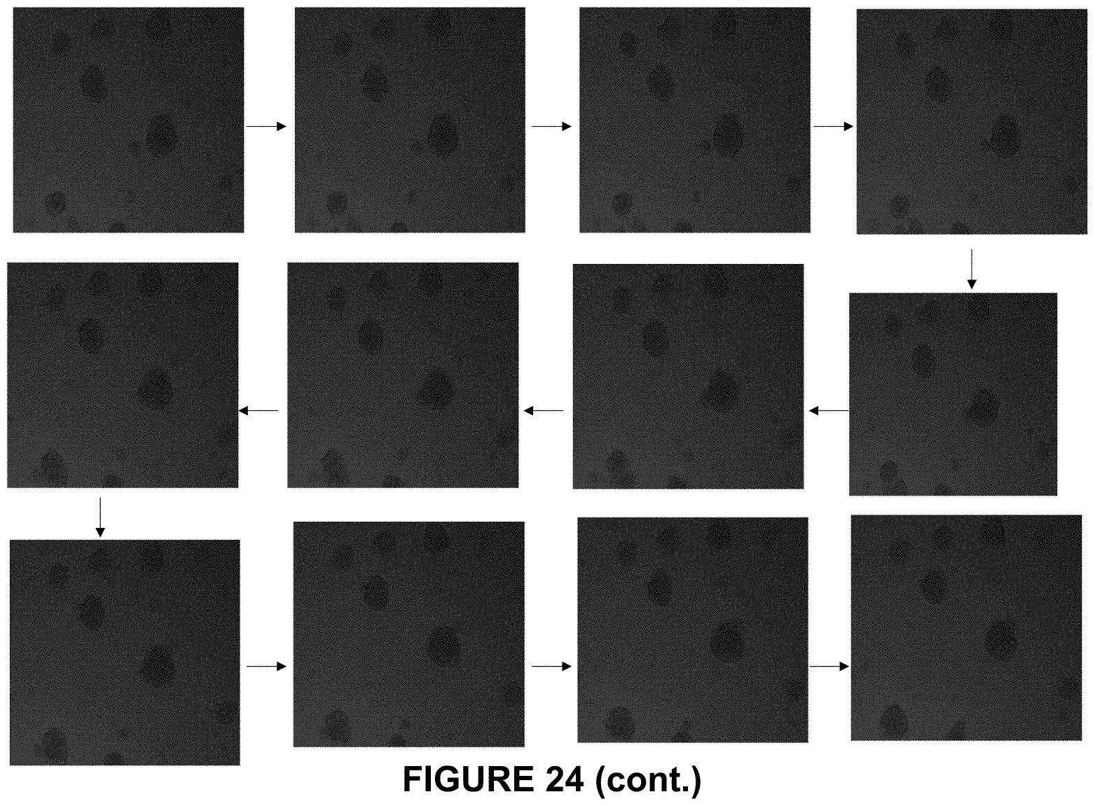

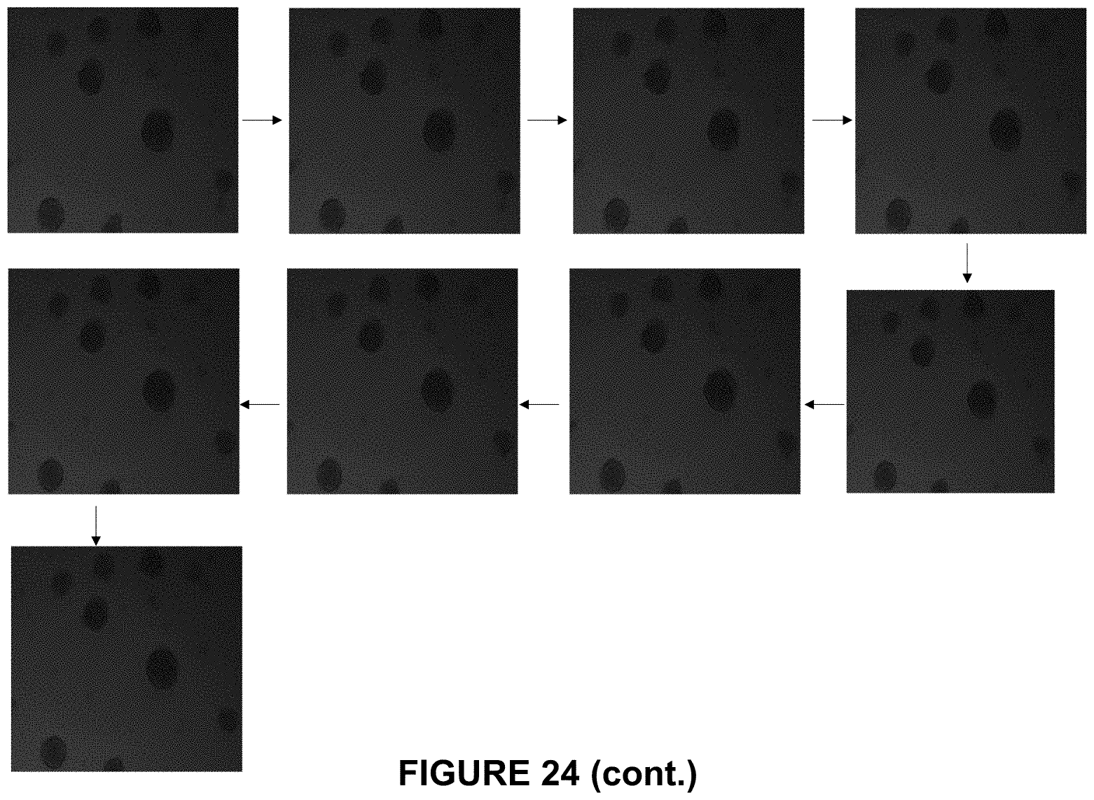

Mouse myoblast muscle cells were then grown on the surface on varying thicknesses cells, forming 3D organoid spheroids on thick CLC surfaces (FIG. 20 through FIG. 24). Cells also consumed the CLC as the control experiment had a micron layer of CLC coating. The cells consumed the liquid crystal and released it from the organoid structures and spread 2-dimensionally.

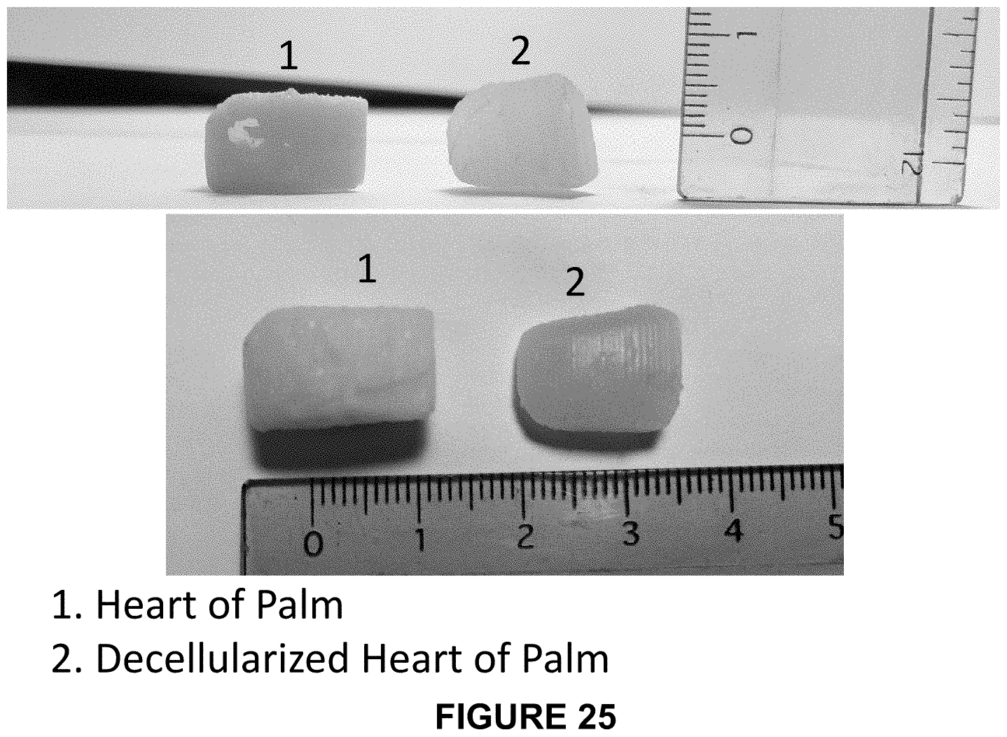

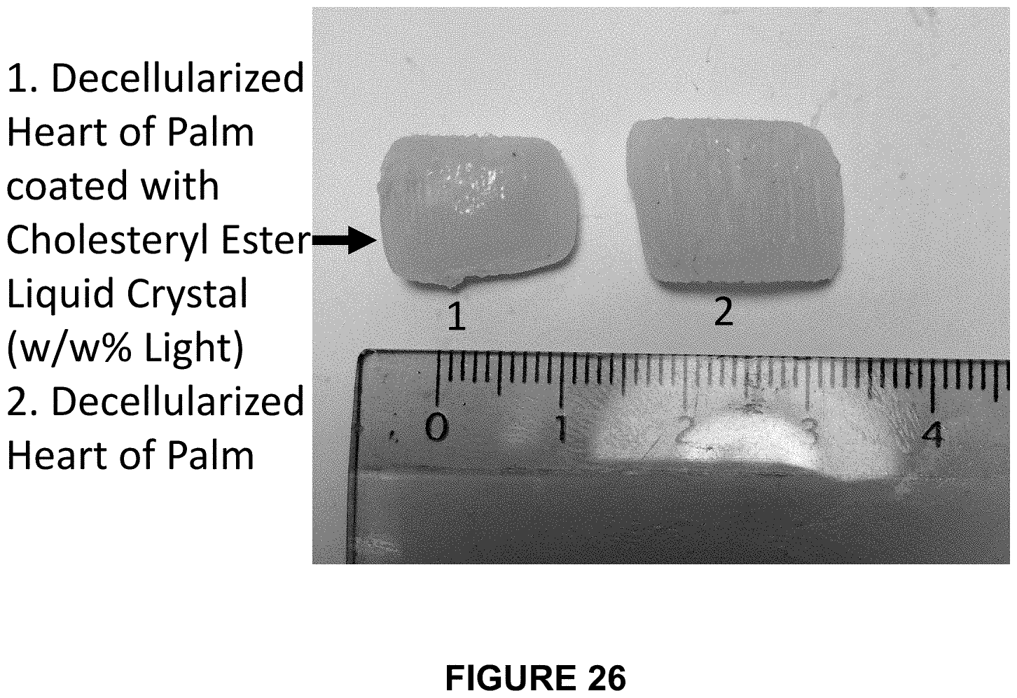

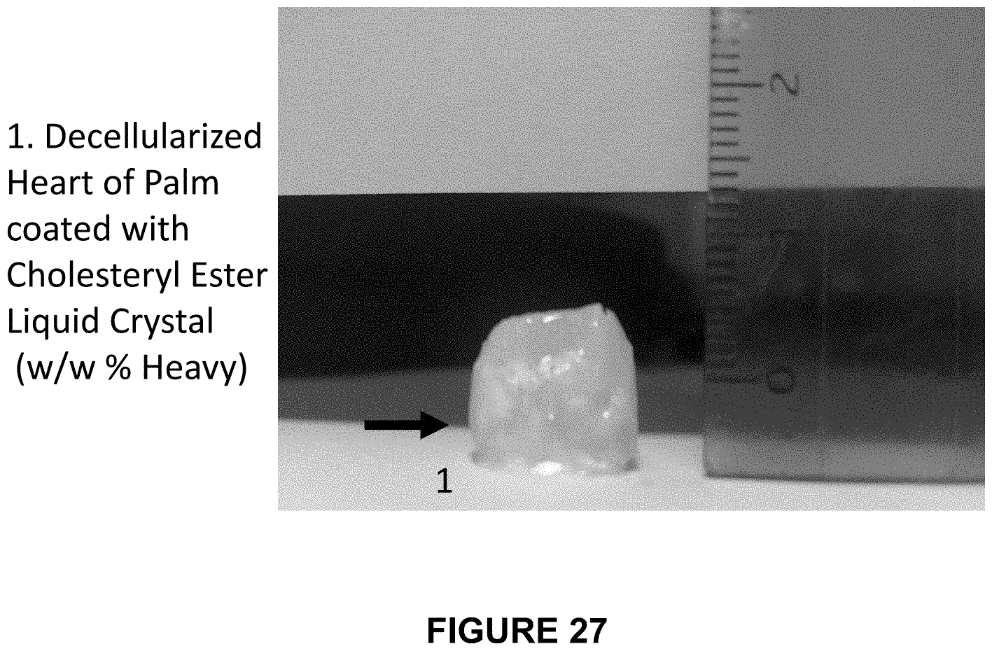

Additional experiments are directed toward the growth of muscle cells with fibroblast to deposit collagen and fat cells to impart flavor. This method develops a more endogenous steak-like tissue. Additional experiments are also directed toward the use of the CLC as a coating material to decellularized plant tissues (e.g., heart of palm) and others (FIG. 25 through FIG. 27).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of inducing cell-culturing, wherein the method comprises culturing at least one cell on a liquid crystal or a composition thereof

2. The method of claim 1, wherein the method comprises culturing the at least one cell on a surface of the liquid crystal or the composition thereof.

3. The method of claim 1, wherein the cell is selected from the group consisting of a tissue cell, epithelial cell, endothelial cell, muscle cell, neuron, adipocyte, and any combination thereof.

4. (canceled)

5. The method of claim 1, wherein the liquid crystal comprises at least one selected from the group consisting of cholesteryl oleyl carbonate, cholesteryl pelargonate, and cholesteryl benzoate.

6. The method of claim 1, wherein the liquid crystal is a cholesteryl ester liquid crystal.

7. The method of claim 1, wherein the composition comprises at least one selected from the group consisting of a polymer, solvent, composite, substrate, and additive.

8. The method of claim 7, wherein the polymer is selected from the group consisting of a fibrous network, edible polymer, food grade polymer, biocompatible polymer, biodegradable polymer, and any combination thereof.

9. (canceled)

10. The method of claim 7, wherein the polymer is selected from the group consisting of polyester, polycaprolactone, polyethylene glycol, and any combination thereof.

11. The method of claim 10, wherein the polymer is polycaprolactone.

12. The method of claim 11, wherein the composition comprises between about 5% to about 75% (w/v) polycaprolactone.

13. The method of claim 7, wherein the composition comprises the polymer and the solvent and wherein the composition is generated by dispersing the liquid crystal and the polymer in the solvent.

14. The method of claim 13, wherein the composition is generated by:
a) dispersing the liquid crystal and the polymer in the solvent; and
b) electrospinning the liquid crystal with the polymer.

15. The method of claim 7, wherein the composition is a cholesteryl ester liquid crystal-based scaffold.

16. The method of claim 15, wherein the composition is a nonwoven cholesteryl ester liquid crystal-based scaffold.

17. The method of claim 16, wherein the composition comprises between about 25% (w/v) to about 50% (w/v) cholesteryl ester liquid crystal.

18. The method of claim 1, wherein the composition is at least one selected from the group consisting of
a) a composition having a mesophase between about 36° C. and about 40° C.;
b) a composition forming striations at a temperature between about 36° C. and about 40° C. and
c) a composition is selected from the group consisting of a pharmaceutical composition, edible composition, scaffold, and any combination thereof.

19. (canceled)

20. The method of claim 1, wherein the liquid crystal or the composition thereof is generated via a solvent-free method, polymer-free method, or a combination thereof

21. A method of exercising at least one cultured cell on a liquid crystal or a composition thereof through a treadmill action.

22. The method of claim 21, wherein the method comprises a stimulus.

23. The method of claim 22, wherein the stimulus is selected from the group consisting of a light stimulus, electrical stimulus, and mechanical stimulus.
